(19) United States
(12) Patent Application Publication
(10) Pub. No.: US 2023/0084638 A1
(43) Pub. Date: Mar. 16, 2023

(54) METHOD AND APPARATUS FOR CLASSIFICATION MODEL TRAINING AND CLASSIFICATION, COMPUTER DEVICE, AND STORAGE MEDIUM

(71) Applicant: Tencent Technology (Shenzhen) Company Limited, Shenzhen (CN)

(72) Inventors: Ziyi YANG, Shenzhen (CN); Zhaofeng Ye, Shenzhen (CN); Benben Liao, Shenzhen (CN); Shengyu Zhang, Shenzhen (CN)

(73) Assignee: Tencent Technology (Shenzhen) Company Limited, Shenzhen (CN)

(21) Appl. No.: 17/984,623

(22) Filed: Nov. 10, 2022

Related U.S. Application Data
(63) Continuation of application No. PCT/CN2022/083074, filed on Mar. 25, 2022.

(30) Foreign Application Priority Data
Apr. 1, 2021 (CN) 2021103551646

(51) Int. Cl. G16B 15/00; G16B 20/30; G16B 40/20

(52) U.S. Cl. CPC G16B 15/00; G16B 20/30; G16B 40/20

(57) ABSTRACT

This disclosure relates to a method and an apparatus for classification model training. The method includes: obtaining a support set and a query set, the support set comprising support sample feature vectors and corresponding drug resistance category labels, and the query set comprising query sample feature vectors and corresponding drug resistance category labels; inputting the support set and the query set into an initial drug resistance classification model; performing drug resistance-related feature screening to obtain target support feature vectors and target query feature vectors; calculating an initial category representation vector corresponding to a drug resistance category; determining training drug resistance category information corresponding to the query sample feature vectors; updating the initial drug resistance classification model based on the training drug resistance category information and the corresponding drug resistance category labels; and obtaining a target drug resistance classification model in response to training being completed.

### RELATED APPLICATION

This application is a continuation application of PCT Patent Application No. PCT/CN2022/083074, filed on Mar. 25, 2022, which claims priority to Chinese Patent Application No. 2021103551646 filed to the China National Intellectual Property Administration on Apr. 1, 2021 and entitled “METHOD AND APPARATUS FOR CLASSIFICATION MODEL TRAINING AND CLASSIFICATION, COMPUTER DEVICE, AND STORAGE MEDIUM”, wherein the content of the of the above-referenced applications is incorporated herein by reference in its entirety.

### FIELD OF THE TECHNOLOGY

This disclosure relates to the field of computer technologies, and in particular, to a method and an apparatus for classification model training and classification, a computer device, and a storage medium.

### BACKGROUND OF THE DISCLOSURE

With the development of artificial intelligence technology, the artificial intelligence technology is used to predict drug resistance classification problems caused by targeted protein mutations. A large quantity of labeled drug resistance classification data caused by targeted protein mutations is obtained for training to obtain an artificial intelligence model, and the artificial intelligence model is used to perform drug resistance classification. Then, due to the difficulty in collecting the drug resistance classification data, a sample size used in training the artificial intelligence model is small, and the feature distribution between data sets is quite different, which makes the trained artificial intelligence model have low accuracy in drug resistance classification.

### SUMMARY

Based on this, to resolve the foregoing technical problems, it is necessary to provide a method and an apparatus for classification model training and classification, a computer device, and a storage medium that can improve the accuracy of drug resistance classification.

In an aspect of the disclosure, a classification model training method is provided. The method may include:

obtaining a support set and a query set, the support set comprising support sample feature vectors and corresponding drug resistance category labels, and the query set comprising query sample feature vectors and corresponding drug resistance category labels;

inputting the support set and the query set into an initial drug resistance classification model;

performing drug resistance-related feature screening on the support sample feature vectors and the query sample feature vectors through the initial drug resistance classification model, to obtain target support feature vectors and target query feature vectors;

calculating an initial category representation vector corresponding to a drug resistance category based on the target support feature vectors;

determining training drug resistance category information corresponding to the query sample feature vectors based on a similarity degree between the target query feature vectors and the initial category representation vector;

updating the initial drug resistance classification model based on the training drug resistance category information and the corresponding drug resistance category labels;

returning to perform the operation of inputting the support set and the query set into the initial drug resistance classification model; and

obtaining a target drug resistance classification model in response to training being completed, the target drug resistance classification model being for identifying a drug resistance category corresponding to protein-compound binding.

In another aspect of the disclosure, a classification model training apparatus is provided. The apparatus includes a memory operable to store computer-readable instructions and a processor circuitry operable to read the computer-readable instructions. When executing the computer-readable instructions, the processor circuitry is configured to:

obtain a support set and a query set, the support set comprising support sample feature vectors and corresponding drug resistance category labels, and the query set comprising query sample feature vectors and corresponding drug resistance category labels;

input the support set and the query set into an initial drug resistance classification model;

perform drug resistance-related feature screening on the support sample feature vectors and the query sample feature vectors through the initial drug resistance classification model, to obtain target support feature vectors and target query feature vectors;

calculate an initial category representation vector corresponding to a drug resistance category based on the target support feature vectors;

determine training drug resistance category information corresponding to the query sample feature vectors based on a similarity degree between the target query feature vectors and the initial category representation vector;

update the initial drug resistance classification model based on the training drug resistance category information and the corresponding drug resistance category labels;

return to perform the operation of inputting the support set and the query set into the initial drug resistance classification model; and

obtain a target drug resistance classification model in response to training being completed, the target drug resistance classification model being for identifying a drug resistance category corresponding to protein-compound binding.

According to the method and the apparatus for classification model training, the support set and the query set are obtained, the support set and the query set are inputted into the initial drug resistance classification model, and the drug resistance-related feature screening is performed based on each support sample feature vector and each query sample feature vector through the initial drug resistance classification model, to obtain each target support feature vector and each target query feature vector, thereby making the features used in training more accurate, and then each target support feature vector is used to calculate the initial category representation vector corresponding to the drug resistance category, which can make the calculated initial category representation vector more accurate. In this case, the similarity degree between each target query feature vector and the initial category representation vector is calculated, thereby determining the training drug resistance category information corresponding to each query sample feature vector, which can make the obtained training drug resistance category information more accurate. Then, the initial drug resistance classification model is updated by using the training drug resistance category information and the corresponding drug resistance category label, and the process returns to perform the step of inputting the support set and the query set into the initial drug resistance classification model. When the training is completed, the target drug resistance classification model is obtained, so that the target drug resistance classification model obtained by training can improve the accuracy of drug resistance classification.

In another aspect of the disclosure, a classification method is provided. The method

may include:

obtaining original classification data and sample data, the original classification data comprising original classification feature vectors, and the sample data comprising sample feature vectors and corresponding sample category labels;

inputting the original classification data and the sample data into a drug resistance classification model;

performing drug resistance-related feature screening based on the original classification feature vectors and the sample feature vectors through the drug resistance classification model, to obtain a target original classification feature vector and target sample feature vectors;

calculating a target category representation vector corresponding to a sample category based on the target sample feature vectors;

determining drug resistance category information corresponding to the original classification feature vector based on a similarity degree between the target original classification feature vector and the target category representation vector; and

outputting the drug resistance category information corresponding to the original classification data through the drug resistance classification model.

According to the method for classification, the original classification data and the sample data are obtained, the original classification data and the sample data are inputted into the drug resistance classification model, and the drug resistance-related feature screening is performed based on the original classification feature vector and each sample feature vector through the drug resistance classification model, to obtain the target original classification feature vector and each target sample feature vector, thereby reducing features unrelated to drug resistance, which makes the obtained target original classification feature vector more accurate. Then, the target category representation vector corresponding to the sample category is calculated based on each target sample feature vector, and the similarity degree between the target original classification feature vector and the target category representation vector, thereby determining the drug resistance category information corresponding to the original classification feature vector. Since the drug resistance classification model is obtained by training by using features related to drug resistance, the drug resistance classification model is used to classify and identify the drug resistance to obtain the drug resistance category information corresponding to the original classification feature vector, which can make the obtained drug resistance category information more accurate.

### BRIEF DESCRIPTION OF THE DRAWINGS

To describe the technical solutions in the embodiments of this disclosure more clearly, the following briefly describes the accompanying drawings required for describing the embodiments. Apparently, the accompanying drawings in the following description show only some embodiments of this disclosure, and a person of ordinary skill in the art may still derive other accompanying drawings according to the accompanying drawings without creative efforts.

FIG. 1 is a diagram of an application environment of a classification model training method in an embodiment.

FIG. 2 is a schematic flowchart of a classification model training method in an embodiment.

FIG. 3 is a schematic flowchart of obtaining a sample feature vector in an embodiment.

FIG. 4 is a schematic flowchart of extracting a query set and a support set in an embodiment.

FIG. 5 is a schematic flowchart of obtaining each target query feature vector in an embodiment.

FIG. 6 is a schematic flowchart of calculating an initial category representation vector in an embodiment.

FIG. 7 is a schematic flowchart of calculating an initial category representation vector in another embodiment.

FIG. 8 is a schematic flowchart of obtaining drug resistance category information in an embodiment.

FIG. 9 is a schematic flowchart of determining training drug resistance category information in an embodiment.

FIG. 10 is a schematic flowchart of determining training drug resistance category information in another embodiment.

FIG. 11 is a schematic diagram of a prototype network in a specific embodiment.

FIG. 12 is a schematic flowchart of obtaining a target drug resistance classification model in an embodiment.

FIG. 13 is a schematic flowchart of a classification method in an embodiment.

FIG. 14 is a schematic flowchart of a classification method in a specific embodiment.

FIG. 15 is a schematic flowchart of a classification model training method in a specific embodiment.

FIG. 16 is an architectural schematic diagram of a classification model in a specific embodiment.

FIG. 17 is a schematic diagram of a test evaluation index in a specific embodiment.

FIG. 18 is a structural block diagram of a classification model training apparatus in an embodiment.

FIG. 19 is a structural block diagram of a classification apparatus in an embodiment.

FIG. 20 is an internal structural diagram of a computer device in an embodiment.

FIG. 21 is an internal structural diagram of a computer device in another embodiment.

### DESCRIPTION OF EMBODIMENTS

To make the objectives, technical solutions, and advantages of this disclosure clearer, the following further describes this disclosure in detail with reference to the accompanying drawings and the embodiments. It is to be understood that the specific embodiments described herein are only used for explaining this disclosure, and are not used for limiting this disclosure.

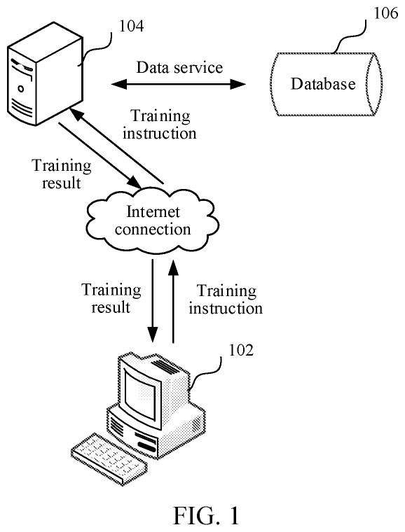

A classification model training method according to this disclosure may be applied to an application environment shown in FIG. 1. A terminal 102 communicates with a server 104 through a network. The server 104 receives a training instruction from the terminal 102. The server 104 obtains a support set and a query set from a database 106 according to the training instruction. The support set includes each support sample feature vector and a corresponding drug resistance category label, and the query set includes each query sample feature vector and a corresponding drug resistance category label. The server 104 inputs the support set and the query set into an initial drug resistance classification model, performs drug resistance-related feature screening on each support sample feature vector and each query sample feature vector through the initial drug resistance classification model, to obtain each target support feature vector and each target query feature vector, calculates an initial category representation vector corresponding to a drug resistance category based on each target support feature vector, and determines training drug resistance category information corresponding to each query sample feature vector based on a similarity degree between each target query feature vector and the initial category representation vector. The server 104 updates the initial drug resistance classification model based on the training drug resistance category information and the corresponding drug resistance category label, returns to perform the step of inputting the support set and the query set into the initial drug resistance classification model, and obtains a target drug resistance classification model when the training is completed. The target drug resistance classification model is used for identifying a drug resistance category corresponding to protein-compound binding. Then, the target drug resistance classification model may be returned to the terminal 102 for display. The terminal 102 may be, but not limited to, a personal computer, a notebook computer, a smartphone, a tablet computer, and a portable wearable device. The server 104 may be implemented by an independent server or a server cluster including a plurality of servers.

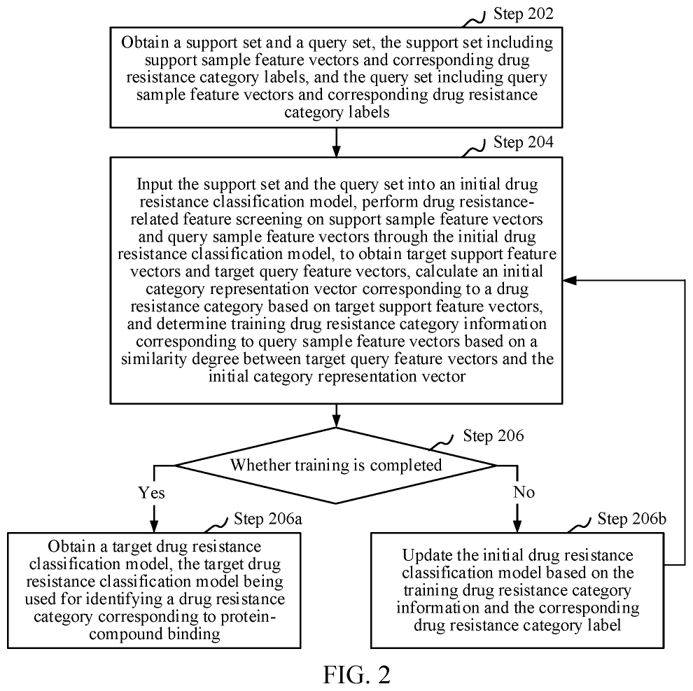

In an embodiment, as shown in FIG. 2, a classification model training method is provided. A description is made using an example in which the method is applied to the server in FIG. 1. It can be understood that the method may also be applied to the terminal, and may further be applied to a system including the terminal and the server, and is implemented by interaction between the terminal and the server. In this embodiment, the method includes the following steps:

Step 202: Obtain a support set and a query set, the support set including each support sample feature vector and a corresponding drug resistance category label, and the query set including each query sample feature vector and a corresponding drug resistance category label.

The support set and the query set are small sample data sets extracted from a sample data set, and the small sample data set usually refers to a sample data set with a quite small sample size, for example, a sample data set with a sample size less than or equal to 30. The sample data set includes each sample feature vector and a drug resistance category label corresponding to each sample feature vector. The drug resistance category label is used for representing the drug resistance category including a drug-resistant category and a non-drug-resistant category. The drug-resistant category refers to that targeted protein after mutation has developed drug resistance to a compound. The non-drug-resistant category refers to that the targeted protein after mutation does not develop the drug resistance to the compound. The support set is a data set used for determining a prototype representation corresponding to each drug resistance category. The query set is a data set used for predicting the drug resistance category. The support sample feature vector refers to a feature vector corresponding to a data sample in the support set. The query sample feature vector refers to a feature vector corresponding to a data sample in the query set.

Specifically, the server may obtain the support set and the query set directly from the database. The server may also obtain the support set and the query set from a server that provides data services. The server may also collect the support set and the query set from the Internet.

In an embodiment, the server may also obtain a small sample data set, and then extract a support set and a query set from the small sample data set in different manners. In an embodiment, the server first extracts a query set from the obtained small sample data set, then determines each sample data similar to the query set from the small sample data set, and then extracts a support set from each similar sample data.

In an embodiment, the server may collect targeted protein data before and after mutation and compound data from the Internet to obtain each sample data, and then extract each sample feature vector from each sample data and collect drug resistance category information to obtain a drug resistance category label, so as to obtain a small sample data set, and then extract a support set and a query set from the small sample data set.

Step 204: Input the support set and the query set into an initial drug resistance classification model, perform drug resistance-related feature screening on each support sample feature vector and each query sample feature vector through the initial drug resistance classification model, to obtain each target support feature vector and each target query feature vector, calculate an initial category representation vector corresponding to a drug resistance category based on each target support feature vector, and determine training drug resistance category information corresponding to each query sample feature vector based on a similarity degree between each target query feature vector and the initial category representation vector.

The initial drug resistance classification model refers to a drug resistance classification model whose model parameters are initialized. The initialization of model parameters may be random initialization, zero initialization, or the like. The drug resistance classification model is used for identifying a drug resistance category corresponding to inputted data, that is, predicting whether the protein after mutation develops the drug resistance to the compound, so as to provide help for doctors to use drugs. The target support feature vector refers to a feature vector obtained by filtering out features unrelated to drug resistance classification and identification in the support feature vectors. The target query feature vector refers to a feature vector obtained by filtering out features unrelated to drug resistance classification and identification in the query feature vectors. The initial category representation vector refers to a prototype representation corresponding to an initial drug resistance category, that is, a center of the category. The training drug resistance category information refers to information of the drug resistance category obtained by identification during training, and each query sample feature vector will identify and obtain a corresponding drug resistance category.

Specifically, the server inputs the support set and the query set into the initial drug resistance classification model, and performs the drug resistance-related feature screening on each support sample feature vector and each query sample feature vector through the initial drug resistance classification model, to obtain each target support feature vector and each target query feature vector, thereby making features in the target support feature vector and the target query feature vector are all features related to identification of the drug resistance category, which is beneficial to improving the identification accuracy of the model. Then, the server uses each target support feature vector to calculate a center of each drug resistance category to obtain the initial category representation vector corresponding to each drug resistance category, finally, calculates the similarity degree between each target query feature vector and the initial category representation vector, and determines the training drug resistance category information corresponding to each query sample feature vector according to the similarity degree. The higher the similarity degree between the target query feature vector and the initial category representation vector, a category to which the target query feature vector belongs is a drug resistance category corresponding to the initial category representation vector.

Step 206: Determine whether training is completed. When the training is completed, step 206a is executed. When the training is not completed, step 206b is executed, and the process returns to step 204 to continue iterative execution.

Step 206a: Obtain a target drug resistance classification model, the target drug resistance classification model being used for identifying a drug resistance category corresponding to protein-compound binding.

Step 206b: Update the initial drug resistance classification model based on the training drug resistance category information and the corresponding drug resistance category label, and return to perform the step of inputting the support set and the query set into the initial drug resistance classification model.

That determining whether the training is completed refers to determining whether the training meets training completion conditions, which include but are not limited to: a quantity of times of training iterations reaching a maximum quantity of times, the model parameters no longer changing, and model loss information reaching a preset threshold. The model loss information refers to an error between a training result and a real result.

Specifically, the server determines whether the training is completed. When the training is not completed, the server calculates and obtains the model loss information based on the training drug resistance category information and the corresponding drug resistance category label, updates parameters in the initial drug resistance classification model reversely by using the model loss information to obtain an updated drug resistance classification model, then uses the updated drug resistance classification model as the initial drug resistance classification model, and returns to iteratively perform the steps of inputting the support set and the query set into the initial drug resistance classification model. Until the training is completed, the server uses an initial drug resistance classification model corresponding to the training completion as the target drug resistance classification model. The target drug resistance classification model is used for identifying a corresponding drug resistance category when the protein after mutation is bound to the compound.

According to the classification model training method, the support set and the query set are obtained, the support set and the query set are inputted into the initial drug resistance classification model, and the drug resistance-related feature screening is performed based on each support sample feature vector and each query sample feature vector through the initial drug resistance classification model, to obtain each target support feature vector and each target query feature vector, thereby making the features used in training more accurate, and then each target support feature vector is used to calculate the initial category representation vector corresponding to the drug resistance category, which can make the calculated initial category representation vector more accurate. In this case, the similarity degree between each target query feature vector and the initial category representation vector is calculated, thereby determining the training drug resistance category information corresponding to each query sample feature vector, which can make the obtained training drug resistance category information more accurate. Then, the initial drug resistance classification model is updated by using the training drug resistance category information and the corresponding drug resistance category label, and the process returns to perform the step of inputting the support set and the query set into the initial drug resistance classification model. When the training is completed, the target drug resistance classification model is obtained, so that the target drug resistance classification model obtained by training can improve the accuracy of drug resistance classification.

In an embodiment, the obtaining a support set and a query set includes:

obtaining a sample data set, the sample data set including a sample feature vector and a drug resistance category label corresponding to each training sample, the sample feature vector being obtained by feature extraction performed based on the training sample, and the training sample including wild-type protein information, mutant protein information, and compound information; and extracting the support set and the query set from the sample data set in different manners.

The sample data set is a set of training sample data. The wild-type protein information refers to specific information of wild-type protein, including but not limited to a structure of the wild-type protein, a physicochemical property of the wild-type protein, and the like. The mutant protein information refers to specific information of mutant protein, including but not limited to a structure of the mutant protein, a physicochemical property of the mutant protein, and the like. The compound information refers to specific information of a small molecular compound that can interact with the wild-type protein and the mutant protein, including a structure of the compound, a physicochemical property of the compound, and the like. Each training sample includes the wild-type protein information, the mutant protein information, and the compound information.

Specifically, the server obtains each training sample, that is, obtains the wild-type protein information, the mutant protein information, and the compound information in each training sample, and then performs the feature extraction on the training sample to obtain the sample feature vector. Features in the extracted sample feature vector include but are not limited to wild-type protein structure features, mutant protein structure features, wild-type protein physicochemical property features, mutant protein physicochemical property features, structure features of crystal protein and compound during interaction, physicochemical property features of compound and residue during interaction, energy features extracted by a scoring function, and the like. In this case, the server obtains the sample data set, and then extracts the training sample in the support set and the training sample in the query set from the sample data set in different manners. The extraction may be performed either with put-back from the sample data set to obtain the support set and the query set, or without put-back from the sample data set to obtain the support set and the query set.

In the above embodiment, the support set and the query set are extracted from the sample data set, and then the model training is performed by using the support set and the query set, which is conducive to improving the classification accuracy of the drug resistance classification model obtained by training.

In an embodiment, after the obtaining a target drug resistance classification model, the method further includes:

using the target drug resistance classification model as the initial drug resistance classification model, returning to perform the step of extracting the support set and the query set from the sample data set, and until a final training completion condition is met, using an initial drug resistance classification model when the final training completion condition is met as a final drug resistance classification model.

The final training completion condition refers to a condition for training to obtain the final drug resistance classification model, including: training times reaching a maximum upper limit of the final training, the parameters of the model no longer changing, or a training error of the model reaching a preset threshold.

Specifically, when the server obtains the target drug resistance classification model, it is still possible to continue training, that is, to use the target drug resistance classification model as the initial drug resistance classification model, and return to perform the step of extracting the support set and the query set from the sample data set. That is, every time the target drug resistance classification model is obtained by training, the server extracts the support set and the query set from the sample data set in different manners, to re-perform the training. Until the final training completion condition is met, the initial drug resistance classification model when the final training completion condition is met is used as the final drug resistance classification model.

In a specific embodiment, an episodic (meta-learning strategy) training strategy may be used for training to obtain the final drug resistance classification model. That is, the strategy is a 2-way k-shot (2 categories, each category has k samples) task of sampling from the sample data set, and each task includes the extracted support set and query set. When all tasks are trained, the final drug resistance classification model is obtained.

In the above embodiment, the support set and the query set are continuously extracted, the support set and the query set are used for training, and the final drug resistance classification model is obtained, thereby improving the generalization ability of the final drug resistance classification model obtained by training.

In an embodiment, as shown in FIG. 3, before the obtaining a sample data set, the method further includes:

Step 302: Obtain a training sample, the training sample including wild-type protein information, mutant protein information, and compound information.

Step 304: Perform wild feature extraction based on the wild-type protein information and the compound information to obtain a wild feature vector.

The wild feature vector refers to a vector of a wild feature extracted by using the wild-type protein information and the compound information. The wild features refer to features corresponding to the wild-type protein information and the compound information, including but not limited to structure features, physicochemical property features, and energy features. The physicochemical property is an index to measure the characteristics of chemical substances, which refers to a physical property and a chemical property. The physical properties include melting and boiling points, a state at room temperature, and a color, and the chemical properties include acidity and alkalinity. The physicochemical property features include physical property features and chemical property features.

Specifically, the server may obtain the training sample from the database, the training sample including the wild-type protein information, the mutant protein information corresponding to the wild-type protein information, and information of the compound that can interact with the wild-type protein and the mutant protein. Then, the server uses the wild-type protein information and the compound information to perform the wild feature extraction, to obtain the wild feature vector. That is, the structure features may be extracted from structure information in the wild-type protein information and the compound information, such as the wild-type protein structure features, compound structure features, and structure features of the wild-type protein and the compound after interaction. The physicochemical property features may be extracted from physicochemical property information in the wild-type protein information and the compound information, such as the wild-type protein physicochemical property features, compound physicochemical property features, and physicochemical property features of the wild-type protein and the compound after interaction. The energy features of the wild-type protein and the compound during interaction may further be extracted by the scoring function. Non-physical energy features may be extracted by an experience-based scoring function, the energy features may also be extracted by an energy function based on physical and empirical potential energy, and the energy features may further be extracted by a knowledge-based scoring function.

Step 306: Perform mutation feature extraction based on the mutant protein information and the compound information to obtain a mutation feature vector.

The mutation feature vector refers to a vector of a mutation feature extracted by using the mutant protein information and the compound information. The mutant features refer to features corresponding to the mutant protein information and the compound information, including but not limited to structure features, physicochemical property features, and energy features.

Specifically, the server uses the mutant protein information and the compound information to perform the mutation feature extraction, to obtain the mutation feature vector. That is, the structure features may be extracted from structure information in the mutant protein information and the compound information, such as the mutant protein structure features, compound structure features, and structure features of the mutant protein and the compound after interaction. The physicochemical property features may be extracted from physicochemical property information in the mutant protein information and the compound information, such as the mutant protein physicochemical property features, compound physicochemical property features, and physicochemical property features of the mutant protein and the compound after interaction. The energy features of the mutant protein and the compound during interaction may further be extracted by the scoring function. Non-physical energy features may be extracted by an experience-based scoring function, the energy features may also be extracted by an energy function based on physical and empirical potential energy, and the energy features may further be extracted by a knowledge-based scoring function.

Step 308: Obtain a sample feature vector corresponding to the training sample based on the wild feature vector and the mutation feature vector.

The sample feature vector refers to a vector of a sample feature corresponding to the training sample.

Specifically, the server uses the extracted wild feature vector and mutation feature vector as the sample feature vector corresponding to the training sample.

In the above embodiment, the wild feature vector and the mutation feature vector are obtained by extraction, and then the sample feature vector corresponding to the training sample is obtained based on the wild feature vector and the mutation feature vector, which makes the obtained sample feature vector more accurate.

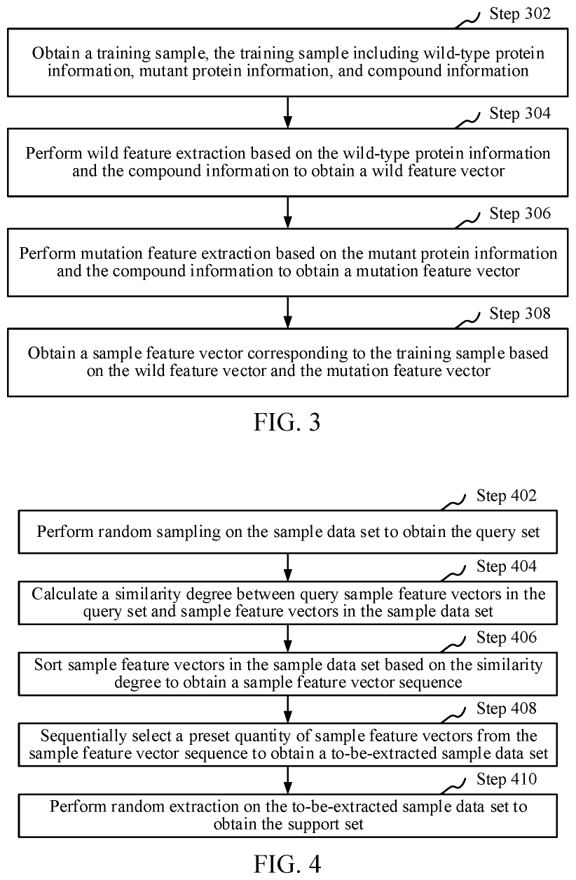

In an embodiment, as shown in FIG. 4, the extracting the support set and the query set from the sample data set includes:

Step 402: Perform sampling on the sample data set to obtain the query set in different manners.

Step 404: Calculate a similarity degree between each query sample feature vector in the query set and each sample feature vector in the sample data set.

The similarity degree is used for representing the similarity between the query sample feature vector and the sample feature vector.

Specifically, the server first extracts the training sample from the sample data set to obtain the query set. Then, the server uses a similarity algorithm to calculate the similarity degree between each query sample feature vector in the query set and each sample feature vector in the sample data set. The similarity algorithm may be a distance similarity algorithm, a cosine similarity algorithm, or the like. The server obtains the similarity degree between each query sample feature vector and each sample feature vector.

Step 406: Sort each sample feature vector in the sample data set based on the similarity degree to obtain a sample feature vector sequence.

The sample feature vector sequence refers to a sequence of the sample feature vectors obtained by sorting according to the similarity degree.

Specifically, the server sorts each sample feature vector in order of the similarity degree from high to low to obtain the sample feature vector sequence, and may also sort each sample feature vector in order of the similarity degree from low to high to obtain the sample feature vector sequence.

Step 408: Sequentially select a preset quantity of sample feature vectors from the sample feature vector sequence to obtain an extraction sample data set.

The extraction sample data set refers to a part of the sample data set used when extracting the support set. The preset quantity refers to a preset quantity of extraction training samples to be selected.

Specifically, the server selects the preset quantity of sample feature vectors from the sample feature vector sequence in order of the similarity degree from high to low to obtain the extraction sample data set. There may also be training samples in which a preset quantity of training samples in the extraction sample data set may be a certain proportion of a total quantity of samples in the sample data set. For example, a preset quantity of 5% of the training samples is extracted as the extraction sample data set.

Step 410: Perform extraction on the extraction sample data set to obtain the support set in different manners.

Specifically, the server extracts the training samples from the extraction sample data set to obtain the support set. The extraction may be with or without put-back extraction.

In an embodiment, the server uses a nonlinear dimensionality reduction algorithm of t-distributed stochastic neighbor embedding (t-SNE) to perform nonlinear dimensionality reduction on the query feature vectors in the query set and the sample feature vectors in the sample data set, to obtain dimensionality-reduced query feature vectors and dimensionality-reduced sample feature vectors. The dimensionality-reduced query feature vectors and the dimensionality-reduced sample feature vectors are used to calculate the similarity degree, which can improve the efficiency of calculating the similarity degree. Then, the extraction sample data set is obtained from the dimensionality-reduced sample feature vectors according to the similarity degree, dimensionality-reduced support feature vectors are extracted from the extraction sample data set, and the dimensionality-reduced support feature vectors and the dimensionality-reduced query feature vectors are used for training of the drug resistance classification model, thereby avoiding the problem of large difference in feature distribution between the data sets, which can improve the accuracy of the drug resistance classification model obtained by training.

In the above embodiment, the query set is extracted first, the similarity degree between the query feature vector in the query set and the sample feature vector in the sample data set is calculated, the preset quantity of sample feature vectors are selected sequentially according to the similarity degree to obtain the extraction sample data set, and then the training samples are extracted from the extraction sample data set to obtain the support set, so that the feature distribution difference between the extracted support set and query set is small, and then the support set and the query set are used for training to obtain the drug resistance classification model, which can improve the classification accuracy of the drug resistance classification model obtained by training.

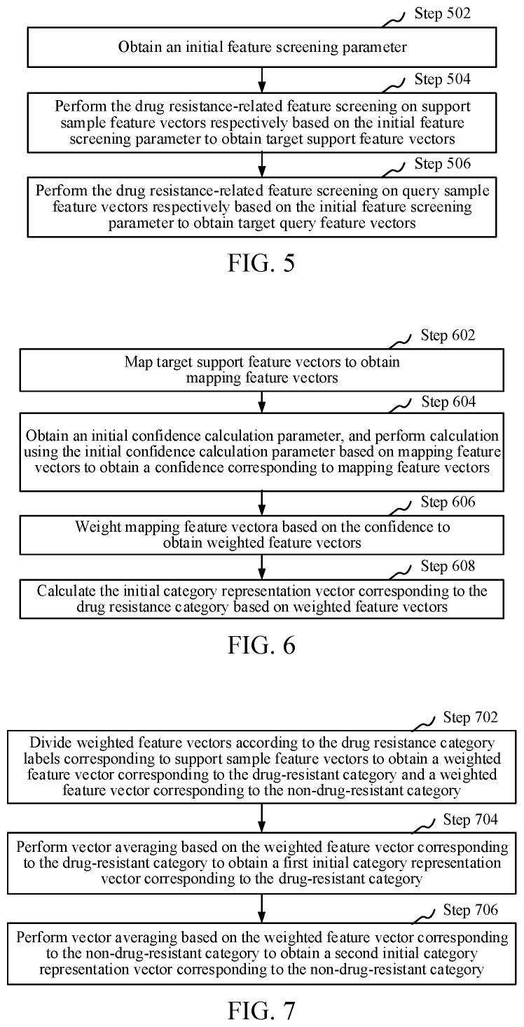

In an embodiment, as shown in FIG. 5, step 204, that is, the performing drug resistance-related feature screening on each support sample feature vector and each query sample feature vector to obtain each target support feature vector and each target query feature vector includes:

Step 502: Obtain an initial feature screening parameter.

Step 504: Perform the drug resistance-related feature screening on each support sample feature vector respectively based on the initial feature screening parameter to obtain each target support feature vector.

A feature screening parameter refers to a parameter used for feature screening, and the feature screening parameter is obtained after updating the initial feature screening parameter by training. The initial feature screening parameter is an initialized feature screening parameter. Different sample features have different feature screening parameters, that is, each sample feature has a corresponding feature screening parameter.

Specifically, the server obtains the initial feature screening parameter, which may be obtained by random initialization, zero initialization, or directly obtained from the database. Then, the server multiplies the initial feature screening parameter with each support sample feature vector, that is, performs the drug resistance-related feature screening to obtain each target support feature vector.

Step 506: Perform the drug resistance-related feature screening on each query sample feature vector respectively based on the initial feature screening parameter to obtain each target query feature vector.

Specifically, the server multiplies the initial feature screening parameter with each query sample feature vector to filter out features unrelated to the drug resistance classification and identification, to obtain each target query feature vector.

In the above embodiment, by using the feature screening parameter to multiply the support sample feature vector and the query sample feature vector, the features unrelated to the drug resistance classification and identification are filtered out to obtain the target support feature vector and the target query feature vector. Then, the target support feature vector and the target query feature vector are used for training of the drug resistance classification model, which can improve the classification accuracy of the drug resistance classification model obtained by training.

In an embodiment, as shown in FIG. 6, step 204, that is, the calculating an initial category representation vector corresponding to a drug resistance category based on each target support feature vector includes:

Step 602: Map each target support feature vector to obtain each mapping feature vector.

The mapping feature vector refers to a vector obtained by mapping the target support feature vector to an embedding space by using an embedding function.

Specifically, the server maps each target support feature vector into the embedding space through the embedding function to obtain each mapping feature vector. The embedding function is obtained by training, which may be an embedding function or the like.

Step 604: Obtain an initial confidence calculation parameter, and perform calculation by using the initial confidence calculation parameter based on each mapping feature vector to obtain a confidence corresponding to each mapping feature vector.

A confidence calculation parameter refers to a parameter that calculates a confidence of the training sample corresponding to the mapping feature vector. Different mapping feature vectors have different confidences, that is, different training samples have different confidences. The confidence is used for representing the credibility of the training sample. The higher the confidence, the better the efficiency of training by using the corresponding training sample. The initial confidence calculation parameter refers to an initialized confidence calculation parameter, which may be obtained by random initialization.

Specifically, the server may obtain the initial confidence calculation parameter directly from the database, may also obtain the initial confidence calculation parameter by the random initialization, and may further obtain the initial confidence calculation parameter provided by a third-party server. Then, the server multiplies each mapping feature vector with the initial confidence calculation parameter to obtain the confidence corresponding to each mapping feature vector. For example, the server may use an adaptive sample weighting strategy of Meta-Weight-Net (MW-Net) to calculate the confidence corresponding to the mapping feature vector. That is, each mapping feature vector is used as an input of MW-Net to output a confidence corresponding to the training sample, in other words, the confidence corresponding to each mapping feature vector.

Step 606: Weight each mapping feature vector based on the confidence to obtain each weighted feature vector.

Specifically, the server uses the confidence to weight each mapping feature vector, that is, by re-weighting the training samples, the training samples may be screened by using the confidence to obtain each weighted feature vector, and the obtained weighted feature vector can better represent the corresponding training sample.

Step 608: Calculate the initial category representation vector corresponding to the drug resistance category based on each weighted feature vector.

Specifically, the server calculates an average vector of each weighted feature vector corresponding to each drug resistance category according to the drug resistance category, and then obtains the initial category representation vector corresponding to each drug resistance category. In an embodiment, a median vector of each weighted feature vector may also be calculated, and the median vector may be used as the initial category representation vector corresponding to the drug resistance category.

In the above embodiment, the confidence corresponding to each mapping feature vector is calculated, then the confidence is used to weight the mapping feature vector to obtain each weighted feature vector, so that the training samples can be screened according to the confidence, and the problem of a noise sample in the training samples is avoided. Then, the weighted feature vector is used to obtain the initial category representation vector, which can improve the accuracy of the obtained initial category representation vector.

In an embodiment, the drug resistance category includes a drug-resistant category and a non-drug-resistant category.

As shown in FIG. 7, step 608, that is, the calculating the initial category representation vector corresponding to the drug resistance category based on each weighted feature vector includes:

Step 702: Divide each weighted feature vector according to the drug resistance category label corresponding to each support sample feature vector to obtain a weighted feature vector corresponding to the drug-resistant category and a weighted feature vector corresponding to the non-drug-resistant category.

Specifically, since the weighted feature vector is obtained based on the support sample feature vector, each weighted feature vector has a corresponding drug resistance category label. The drug resistance category labels include a label corresponding to the drug-resistant category and a label corresponding to the non-drug-resistant category. The server divides each weighted feature vector according to the drug resistance category label corresponding to each support sample feature vector, and then obtains each weighted feature vector corresponding to the drug-resistant category and each weighted feature vector corresponding to the non-drug-resistant category.

Step 704: Perform vector averaging based on the weighted feature vector corresponding to the drug-resistant category to obtain a first initial category representation vector corresponding to the drug-resistant category.

The first initial category representation vector is a vector for representing the drug-resistant category.

Specifically, the server calculates an average vector of each weighted feature vector corresponding to the drug-resistant category, and uses the average vector as the first initial category representation vector corresponding to the drug-resistant category.

Step 706: Perform vector averaging based on the weighted feature vector corresponding to the non-drug-resistant category to obtain a second initial category representation vector corresponding to the non-drug-resistant category.

The second initial category representation vector is a vector for representing the non-drug-resistant category.

Specifically, the server calculates an average vector of each weighted feature vector corresponding to the non-drug-resistant category, and uses the average vector as the second initial category representation vector corresponding to the non-drug-resistant category. In a specific embodiment, drug-resistant means that a relative binding free energy difference between a compound (ligand) and a wild-type and mutant protein target (receptor) is greater than 1.36 kcal/mol. Non-resistant means that the relative binding free energy difference between the compound (ligand) and the wild-type and mutant protein target (receptor) is less than 1.36 kcal/mol.

In the above embodiment, each weighted feature vector corresponding to the drug-resistant category label is averaged to obtain the first initial category representation vector, and each weighted feature vector corresponding to the non-drug-resistant category label is averaged to obtain the second initial category representation vector, which can improve the accuracy of the obtained initial category representation vector and facilitate subsequent use.

In an embodiment, as shown in FIG. 8, step 204, that is, the determining training drug resistance category information corresponding to each query sample feature vector based on a similarity degree between each target query feature vector and the initial category representation vector includes:

Step 802: Calculate a distance between a current target query feature vector and the first initial category representation vector and a distance between the current target query feature vector and the second initial category representation vector, to obtain a current first initial distance and a current second initial distance.

The current target query feature vector refers to a target query feature vector whose similarity degree needs to be calculated currently, and each target query feature vector may be sequentially used as the current target query feature vector. The current first initial distance refers to a similarity distance between the current target query feature vector and the first initial category representation vector. The current second initial distance refers to a similarity distance between the current target query feature vector and the second initial category representation vector.

Specifically, the server uses the distance similarity algorithm to calculate a distance between the current target query feature vector and the first initial category representation vector to obtain the current first initial distance, and to calculate a distance between the current target query feature vector and the second initial category representation vector to obtain the current second initial distance. The distance similarity algorithm may be the Euclidean distance algorithm, or the like.

Step 804: Compare the current first initial distance with the current second initial distance, training drug resistance category information corresponding to the current target query feature vector is the non-drug-resistant category when the current first target distance exceeds the current second target distance, and the training drug resistance category information corresponding to the current target query feature vector is the drug-resistant category when the current first target distance does not exceed the current second target distance.

Specifically, the server compares a size of the current first initial distance with a size of the current second initial distance. When the current first target distance exceeds the current second target distance, it indicates that a distance between the current target query feature vector and the second initial category representation vector is close, and the training drug resistance category information corresponding to the current target query feature vector is the non-drug-resistant category. When the current first target distance does not exceed the current second target distance, it indicates that a distance between the current target query feature vector and the first initial category representation vector is close, and the training drug resistance category information corresponding to the current target query feature vector is the drug-resistant category.

In the above embodiment, the distance between the target query feature vector and the initial category representation vector is calculated, and then the drug resistance category corresponding to the target query feature vector is determined according to the distance, thereby improving the accuracy of the obtained drug resistance category.

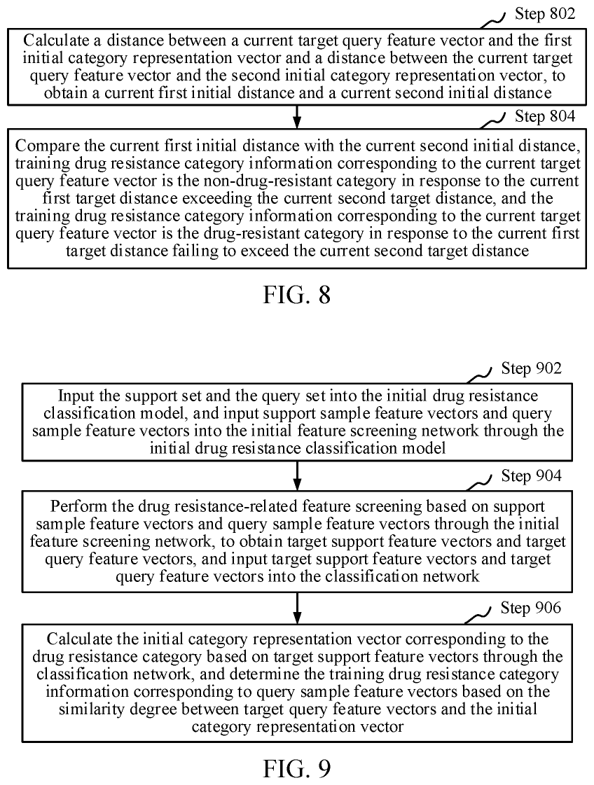

In an embodiment, the initial drug resistance classification model includes an initial feature screening network and an initial classification network. As shown in FIG. 9, step 204, that is, the inputting the support set and the query set into an initial drug resistance classification model includes:

Step 902: Input the support set and the query set into the initial drug resistance classification model, and input each support sample feature vector and each query sample feature vector into the initial feature screening network through the initial drug resistance classification model.

The initial feature screening network refers to a feature screening network whose network parameters are initialized, and the feature screening network is a network used for filtering out features unrelated to the drug resistance classification and identification. The initial classification network is an initialized classification network, and the classification network is a network used for classifying and identifying the drug resistance.

Specifically, the server inputs the support set and the query set into the initial drug resistance classification model, that is, inputs each support sample feature vector and each query sample feature vector into the initial feature screening network in the initial drug resistance classification model for feature screening.

Step 904: Perform the drug resistance-related feature screening based on each support sample feature vector and each query sample feature vector through the initial feature screening network, to obtain each target support feature vector and each target query feature vector, and input each target support feature vector and each target query feature vector into the classification network.

Specifically, the drug resistance-related feature screening is performed through the initial feature screening network, that is, by performing the drug resistance-related feature screening on each support sample feature vector, each target support feature vector is obtained, and by performing the drug resistance-related feature screening on each query sample feature vector, each target query feature vector is obtained. Then, each target support feature vector and each target query feature vector are inputted into the classification network. In a specific embodiment, the feature screening network may be a softmax (Logistic Regression) network, and the drug resistance-related feature screening is performed using an initial softmax network. That is, the feature screening may be performed using formula (1) as shown below.

$${x}_{new} = β ( θ ) ex @ {f}_{θ} ( x ) \\ {β}_{i} ( θ ) = \frac{{exp ( θ )}_{i}}{{\sum }_{j} {exp ( θ )}_{j}} \\ {\sum }_{i} {β}_{i} ( θ ) = 1 \qquad Formula ( 1 )$$

Where f represents a softmax network, θ represents a feature screening network parameter, x refers to an inputted feature vector, and x_{new} refers to an outputted feature vector. x_{new}=β(θ)e X represents that the inputted feature vector is multiplied by a position element corresponding to the feature screening network parameter, exp refers to an exponential operator calculated according to elements, i refers to an i-th inputted feature vector, and j refers to a total quantity of inputted feature vectors

$${\sum }_{i} {β}_{i} ( θ ) = 1$$

represents that a sum of all

normalized network parameter vectors is 1.

$${β}_{i} ( θ ) = \frac{{exp ( θ )}_{i}}{{\sum }_{j} {exp ( θ )}_{j}}$$

represents the normalization of the network parameter vectors.

Step 906: Calculate the initial category representation vector corresponding to the drug resistance category based on each target support feature vector through the classification network, and determine the training drug resistance category information corresponding to each query sample feature vector based on the similarity degree between each target query feature vector and the initial category representation vector.

Specifically, the inputted each target support feature vector and each target query feature vector are obtained through the classification network, average calculation is performed on the target support feature vectors corresponding to different drug resistance category labels to obtain initial category representation vectors corresponding to different drug resistance categories, then the similarity degree between each target query feature vector and the initial category representation vector is calculated, and the training drug resistance category information corresponding to each query sample feature vector is determined according to the similarity degree.

In a specific embodiment, the category representation vector may be calculated using formula (2) as shown below.

$${C}_{n} = \frac{1}{\vert {S}_{n} \vert} {\sum }_{( {x}_{i} , {y}_{i} ) ∈ {S}_{n}} {g}_{ϕ} ( {f}_{θ} ( {x}_{i} ) ) \qquad Formula ( 2 )$$

Where Cₙ represents a category representation vector, and n represents a category. In this disclosure, n∈{0,1} is discrete. Sₙ represents each support feature vector corresponding to the drug resistance category n. yᵢ represents a drug resistance category label corresponding to an i-th support feature vector. xⁱ. represents the i-th support feature vector. f_{θ}(xᵢ) represents a target support feature vector outputted through a softmax network layer based on the i-th support feature vector x, g represents an embedding function, and Φ refers to a mapping parameter.

Then, the similarity degree between each target query feature vector and the initial category representation vector may be calculated using formula (3) shown below to determine the training drug resistance category information corresponding to each query sample feature vector.

$${p}_{ϕ , θ} ( y \vert x , S ) = \frac{exp ( - d ( {g}_{ϕ} ( {f}_{θ} ( {x}_{i} ) ) , {C}_{n} ) )}{{\sum }_{{n}^{′}} exp ( - d ( {g}_{ϕ} ( {f}_{θ} ( {x}_{i} ) ) , {C}_{{n}^{′}} ) )} \qquad Formula ( 3 )$$

Where P_{Φ, θ}(y|x, S) refers to a probability that a query sample feature vector x in the query set outputted through the classification network belongs to a category y. S refers to the support set. d (g_{Φ}(f_{θ}(xᵢ)), Cₙ) represents a similarity degree between the query sample feature vector x and the category representation vector.

In the above embodiment, the features unrelated to the drug resistance classification and identification are filtered out through the initial feature screening network, to obtain the target support feature vector. Then, the initial category representation vector corresponding to the drug resistance category is calculated by using the initial classification network, and the training drug resistance category information corresponding to each query sample feature vector is determined based on the similarity degree between each target query feature vector and the initial category representation vector, so that the obtained training drug resistance category information is more accurate.

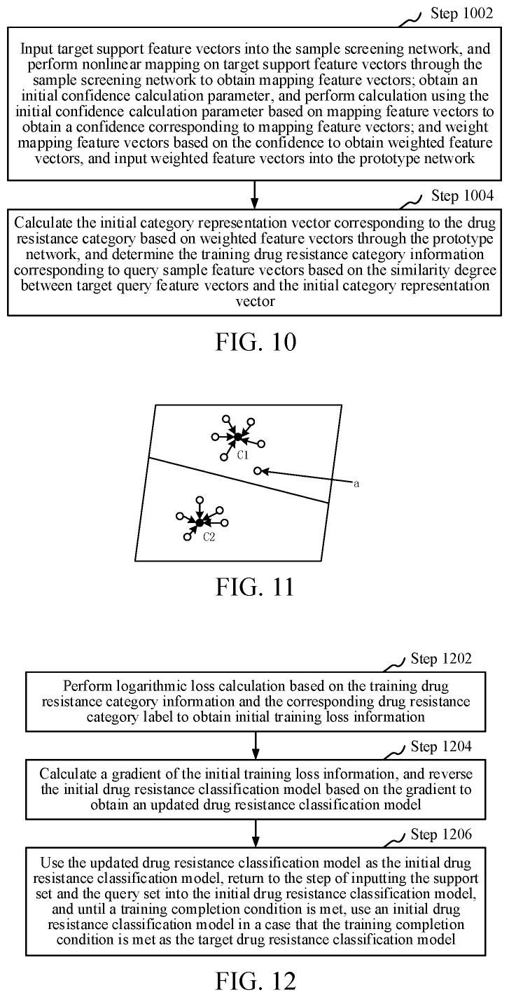

In an embodiment, the classification network includes a sample screening network and a prototype network. As shown in FIG. 10, step 904, that is, the inputting each target support feature vector and each target query feature vector into the classification network includes:

Step 1002: Input each target support feature vector into the sample screening network, and map each target support feature vector through the sample screening network to obtain each mapping feature vector; obtain an initial confidence calculation parameter, and perform calculation by using the initial confidence calculation parameter based on each mapping feature vector to obtain a confidence corresponding to each mapping feature vector; and weight each mapping feature vector based on the confidence to obtain each weighted feature vector, and input each weighted feature vector into the prototype network.

The sample screening network is a network that screens the inputted training samples.

Specifically, the server inputs each target support feature vector into the sample screening network, and maps each target support feature vector into the embedding space through the sample screening network to obtain each mapping feature vector. Then, the server obtains the initial confidence calculation parameter in the sample screening network, and calculates a product of each mapping feature vector and the initial confidence calculation parameter, to obtain the confidence corresponding to each mapping feature vector. Then, the server uses the confidence to weight each mapping feature vector to obtain each weighted feature vector, and finally, inputs each weighted feature vector into the prototype network. In a specific embodiment, the server may use formula (4) as shown below to weight the feature vector.

vᵢ·g_{Φ}(f_{θ}(xᵢ)) Formula (4)

Where vᵢ represents a confidence corresponding to a support feature vector in an i-th th support set, vᵢ∈[0,1] represents that the confidence is in a range of 0 to 1. g_{Φ}(f_{θ}(xᵢ)) represents an i-th mapping feature vector. A product of the confidence and the mapping feature vector is calculated to obtain the weighted feature vector. The mapping feature vector may be inputted into MW-Net to obtain an outputted confidence, that is, the confidence may be calculated by using formula (5) as shown below.

vᵢ=v(g_{Φ}(f_{Φ}(xᵢ))Θ) Formula (5)

Where V represents a confidence calculation network in the sample screening network, and e represents a confidence calculation parameter in the sample screening network.

Step 1004: Calculate the initial category representation vector corresponding to the drug resistance category based on each weighted feature vector through the prototype network, and determine the training drug resistance category information corresponding to each query sample feature vector based on the similarity degree between each target query feature vector and the initial category representation vector.

Specifically, the server uses each weighted feature vector to calculate the initial category representation vector corresponding to the drug resistance category through the prototype network, calculates the similarity degree between each target query feature vector and the initial category representation vector, and obtains the training drug resistance category information corresponding to each query sample feature vector according to the similarity degree.

In a specific embodiment, the server may calculate and obtain the category representation vector by using formula (6) as shown below.

$${C}_{n} = \frac{1}{\vert {S}_{n} \vert} {\sum }_{( {x}_{i} , {y}_{i} ) ∈ {S}_{n}} V ( {g}_{ϕ} ( {f}_{θ} ( {x}_{i} ) ) ; Θ ) \cdot {g}_{ϕ} ( {f}_{θ} ( {x}_{i} ) ) \qquad Formula ( 6 )$$

Where v(g_{Φ}(f_{Φ}(f_{θ}(xᵢ)); Θ)·g_{Φ}(f_{θ}(xᵢ)) represents an i-th weighted feature vector. Then, the server may calculate and obtain the training drug resistance category information corresponding to the query sample feature vector by using formula (7) as shown below.

$${p}_{ϕ , θ , Θ} ( y \vert x , S ) = \frac{exp ( - d ( {g}_{ϕ} ( {f}_{θ} ( {x}_{i} ) ) , {C}_{n} ) )}{{\sum }_{{n}^{′}} exp ( - d ( {g}_{ϕ} ( {f}_{θ} ( {x}_{i} ) ) , {C}_{{n}^{′}} ) )} \qquad Formula ( 7 )$$

Where Φ refers to a mapping parameter by which the feature vector is mapped into the embedding space, θ represents the feature screening network parameter, and Θ represents the confidence calculation parameter in the sample screening network. As shown in FIG. 11, which is a schematic diagram of the category representation vector, a category representation vector C1 corresponding to the drug-resistant category and a category representation vector C2 corresponding to the non-drug-resistant category obtained by calculation through the support set of small samples, and then, training drug resistance category information corresponding to a query feature vector a in the query set is calculated. If a similarity degree between a target query feature vector corresponding to the query feature vector and the category representation vector C1 is high, the training drug resistance category information corresponding to the query feature vector a is the drug-resistant category.

In the above embodiment, the sample screening is performed according to the confidence through the sample screening network to obtain each weighted feature vector, finally, each weighted feature vector is inputted into the prototype network, then the category representation vector is calculated through the prototype network, and the corresponding training drug resistance category information is determined through the similarity degree between the category representation vector and each query sample feature vector, which can reduce noise data and improve the accuracy of the obtained training drug resistance category information.

In an embodiment, as shown in FIG. 12, step 206b, that is, the updating the initial drug resistance classification model based on the training drug resistance category information and the corresponding drug resistance category label, returning to perform the step of inputting the support set and the query set into the initial drug resistance classification model, and obtaining a target drug resistance classification model when the training is completed includes:

Step 1202: Perform logarithmic loss calculation based on the training drug resistance category information and the corresponding drug resistance category label to obtain initial training loss information.

The initial training loss information refers to an error between training drug resistance category information obtained by calculation during initial training and a corresponding drug resistance category label.

Specifically, the server uses a logarithmic loss function to calculate the error between the training drug resistance category information and the corresponding drug resistance category label to obtain the initial training loss information.

Step 1204: Calculate a gradient of the initial training loss information, and reverse the initial drug resistance classification model based on the gradient to obtain an updated drug resistance classification model.

Step 1206: Use the updated drug resistance classification model as the initial drug resistance classification model, return to the step of inputting the support set and the query set into the initial drug resistance classification model, and until a training completion condition is met, use an initial drug resistance classification model when the training completion condition is met as the target drug resistance classification model.

Specifically, the server uses a gradient descent algorithm to reversely update the initial drug resistance classification model. It may be determined first whether the training completion condition is met, for example, it is possible to compare whether the initial training loss information reaches a preset loss threshold, if not, it indicates that the training is not completed. In this case, the gradient is calculated using the initial training loss information, parameters in the initial drug resistance classification model are updated reversely based on the gradient, and when the update is completed, the updated drug resistance classification model is obtained. Then, the updated drug resistance classification model is used as the initial drug resistance classification model, the process returns to perform the step of inputting the support set and the query set into the initial drug resistance classification model, and until the training completion condition is met, the initial drug resistance classification model when the training completion condition is met is used as the target drug resistance classification model.

In a specific embodiment, the initial drug resistance classification model is updated using formula (8) shown below as a loss function.

$$ϕ , θ , Θ = {arg min}_{ϕ , θ , Θ} {E}_{d ⊂ {D}_{s}} {\sum }_{( x , y ) ∈ Q} - log {p}_{ϕ , θ , Θ} ( y \vert x , S ) \qquad Formula ( 8 )$$

Where Dₛ={xᵢ, yᵢ}ᵢ₌₁^{N} represents a training sample set, xᵢ∈X represents a training sample, and X represents a training sample space. yᵢ∈Y represents a drug resistance category label corresponding to the training sample, and Y represents a label space.

N is a quantity of all training samples. Each training sample xᵢ is a D-dimensional sample feature vector, and yᵢ∈{0,1} is discrete. S refers to a support set and Q refers to a query set. E_{d⊂D}_{ₛ} refers to a task of extracting the support set and the query set from the training sample set for training every time, that is, each n-way k-shot task is defined as an episodic d=(S; Q). That is, parameters Φ, θ, Θ in the drug resistance classification model are updated by the above loss function. Until the parameters Φ, θ, Θ are minimized, the obtained parameters Φ, θ, Θ are used as parameters in the final drug resistance classification model.

In the above embodiment, the logarithmic loss calculation is performed on the training drug resistance category information and the corresponding drug resistance category label to obtain the initial training loss information, then the initial training loss information is used to reversely update the initial drug resistance classification model, and the process returns to perform the step of inputting the support set and the query set into the initial drug resistance classification model. Until the training completion condition is met, the initial drug resistance classification model when the training completion condition is met is used as the target drug resistance classification model. As a result, the accuracy of the target drug resistance classification model obtained by training is ensured.

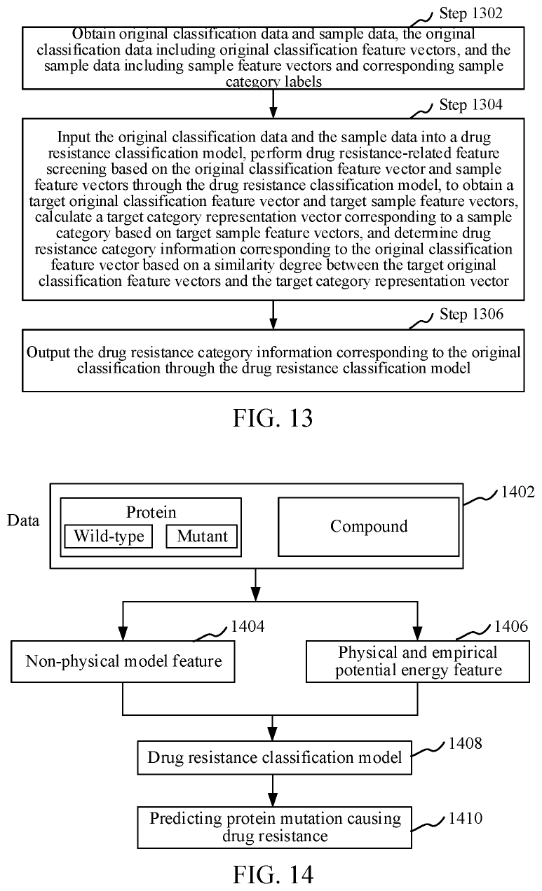

In an embodiment, as shown in FIG. 13, a classification method is provided. A description is made using an example in which the method is applied to the server in FIG. 1. It can be understood that the method may also be applied to the server, and may further be applied to a system including the terminal and the server, and is implemented by interaction between the terminal and the server. In this embodiment, the method includes the following steps:

Step 1302: Obtain original classification data and sample data, the original classification data including original classification feature vectors, and the sample data including each sample feature vector and a corresponding sample category label.

The original classification data refers to data that needs to be classified, and the original classification feature vector refers to a feature vector that needs to be subjected to the drug resistance category identification. The sample category label refers to a label corresponding to the drug resistance category. The sample feature vector refers to a feature vector corresponding to the training sample.

Specifically, the server may directly obtain the original classification data and the sample data from the database. The server may also obtain wild-type protein information, mutant protein information and compound information that need to be subjected to the drug resistance classification, and then extract original classification features corresponding to the wild-type protein information, the mutant protein information and the compound information, to obtain the original classification feature vectors. In a specific embodiment, the wild-type protein structure features, the mutant protein structure features, the wild-type protein physicochemical property features, the mutant protein physicochemical property features, the structure features of crystal protein and compound during interaction, the physicochemical property features of compound and residue during interaction, and the energy features extracted by the scoring function are extracted from the wild-type protein information, the mutant protein information and the compound information, to obtain the wild-type feature vectors and the mutant feature vectors, and then differences between the wild-type feature vectors and the mutant feature vectors are calculated to obtain the original classification feature vectors. Then, each sample feature vector and the corresponding sample category label are obtained from the database. The server may also obtain the original classification data from the terminal, and then find each sample feature vector and the corresponding sample category label from the database.

Step 1304: Input the original classification data and the sample data into a drug resistance classification model, perform drug resistance-related feature screening based on the original classification feature vector and each sample feature vector through the drug resistance classification model, to obtain a target original classification feature vector and each target sample feature vector, calculate a target category representation vector corresponding to a sample category based on each target sample feature vector, and determine drug resistance category information corresponding to the original classification feature vector based on a similarity degree between the target original classification feature vector and the target category representation vector.

The drug resistance classification model may be a model obtained by training in any one of the embodiments of the drug resistance classification model training method.

Specifically, the server deploys the trained drug resistance classification model to a server. When receiving the original classification data and the sample data, the server inputs the original classification data and the sample data into the drug resistance classification model for drug resistance classification and identification. That is, the drug resistance-related feature screening is performed based on the original classification feature vector and each sample feature vector through the drug resistance classification model, to obtain the target original classification feature vector and each target sample feature vector. The target category representation vector corresponding to the sample category is calculated based on each target sample feature vector, and the drug resistance category information corresponding to the original classification feature vector is determined based on the similarity degree between the target original classification feature vector and the target category representation vector.

In an embodiment, the original classification data and the sample data are inputted into the drug resistance classification model, the drug resistance classification model performs the drug resistance-related feature screening on the original classification feature vector and each sample feature vector through the feature screening network, to obtain the target original classification feature vector and each target sample feature vector, and then each target sample feature vector is mapped through the sample screening network to obtain each mapping feature vector. The confidence calculation parameter is obtained, and calculation is performed by using the confidence calculation parameter based on each mapping feature vector to obtain the confidence corresponding to each mapping feature vector. Each mapping feature vector is weighted based on the confidence to obtain each weighted feature vector, and each weighted feature vector is inputted into the prototype network. The category representation vector corresponding to the drug resistance category is calculated based on each weighted feature vector through the prototype network, the similarity degree between the target original classification feature vector and the category representation vector is calculated, and the training drug resistance category information corresponding to each query sample feature vector is determined according to the similarity degree.

Step 1306: Output the drug resistance category information corresponding to the original classification data through the drug resistance classification model.

Specifically, the drug resistance classification model in the server outputs the obtained drug resistance category information corresponding to the original classification data, thereby obtaining the drug resistance category information corresponding to the original classification data, and then returns the drug resistance category information to the terminal for display.

In an embodiment, each target weighted feature vector is divided according to the sample category label corresponding to each sample feature vector through the drug resistance classification model in the server, to obtain a target weighted feature vector corresponding to the drug-resistant category and a target weighted feature vector corresponding to the non-drug-resistant category. The vector averaging is performed based on the target weighted feature vector corresponding to the drug-resistant category to obtain a first target category representation vector corresponding to the drug-resistant category. The vector averaging is performed based on the target weighted feature vector corresponding to the non-drug-resistant category to obtain a second target category representation vector corresponding to the non-drug-resistant category. Then, a distance between the target original classification feature vector and the first target category representation vector and a distance between the target original classification feature vector and the second target category representation vector are calculated, to obtain a first target distance and a second target distance. Comparing the first target distance with the second target distance, when the first target distance exceeds the second target distance, the drug resistance category information corresponding to the original classification feature vector is the non-drug-resistant category, and when the first target distance does not exceed the second target distance, the drug resistance category information corresponding to the original classification feature vector is the drug-resistant category.

According to the classification method, the original classification data and the sample data are obtained, the original classification data and the sample data are inputted into the drug resistance classification model, and the drug resistance-related feature screening is performed based on the original classification feature vector and each sample feature vector through the drug resistance classification model, to obtain the target original classification feature vector and each target sample feature vector, thereby reducing features unrelated to drug resistance, which makes the obtained target original classification feature vector more accurate. Then, the target category representation vector corresponding to the sample category is calculated based on each target sample feature vector, and the similarity degree between the target original classification feature vector and the target category representation vector, thereby determining the drug resistance category information corresponding to the original classification feature vector. Since the drug resistance classification model is obtained by training by using features related to drug resistance, the drug resistance classification model is used to classify and identify the drug resistance to obtain the drug resistance category information corresponding to the original classification feature vector, which can make the obtained drug resistance category information more accurate.

In a specific embodiment, as shown in FIG. 14, a specific flowchart of the drug resistance classification method is shown, to be specific: the server obtains original classification data uploaded by the terminal (1402), the original classification data including original classification features corresponding to the wild-type protein information, the mutant protein information and the compound information, obtains the original classification feature vectors, and then performs non-physical model feature extraction on the original classification data, that is, extraction of the structure features, the physicochemical property features, and the energy features extracted by the experience-based scoring function (1404). The server performs physical and empirical potential energy feature extraction on the original classification data, that is, calculates the energy features by Rosetta, which is a hybrid physical and empirical potential energy modeling program, to obtain the wild-type feature vector and the mutant feature vector (1406). The server calculates the difference between the wild-type feature vector and the mutant feature vector to obtain the original classification feature vector. Then, the server obtains the sample data from the database, and inputs the original classification feature vector, each sample feature vector and the corresponding sample category label into the trained drug resistance classification model for drug resistance prediction, to obtain the outputted drug resistance category information (1408). The server determines, according to the drug resistance category information, whether the mutant protein obtained after protein mutation based on the wild-type protein information develops drug resistance when bonded to the compound (1410).

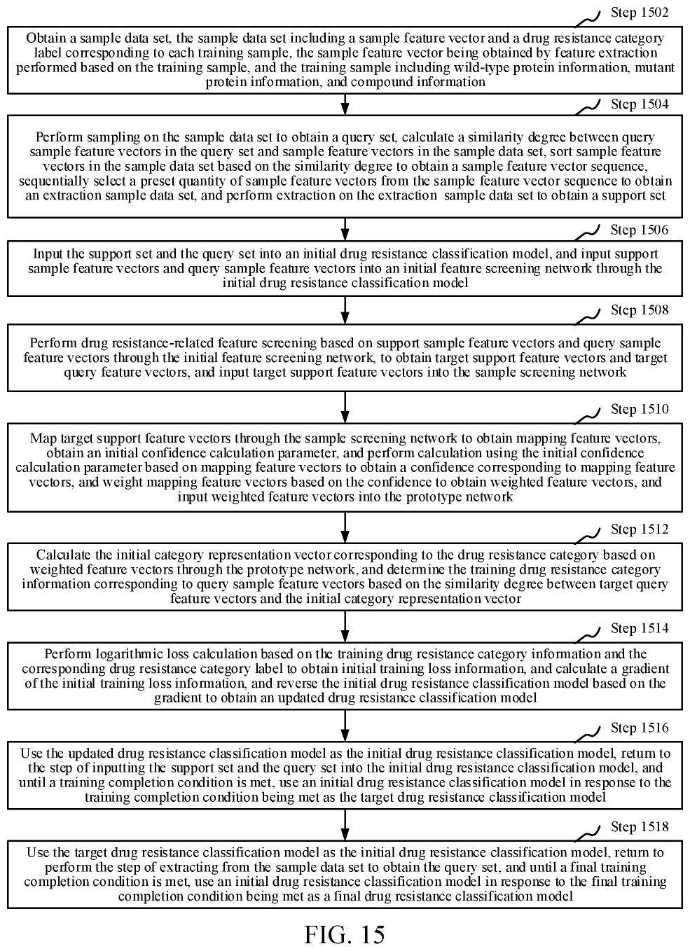

In a specific embodiment, as shown in FIG. 15, a classification model training method is provided, which is performed in the server and specifically includes the following steps.

Step 1502: Obtain a sample data set, the sample data set including a sample feature vector and a drug resistance category label corresponding to each training sample, the sample feature vector being obtained by feature extraction performed based on the training sample, and the training sample including wild-type protein information, mutant protein information, and compound information.

Step 1504: Perform sampling on the sample data set to obtain a query set, calculate a similarity degree between each query sample feature vector in the query set and each sample feature vector in the sample data set, sort each sample feature vector in the sample data set based on the similarity degree to obtain a sample feature vector sequence, sequentially select a preset quantity of sample feature vectors from the sample feature vector sequence to obtain an extraction sample data set, and perform extraction on the extraction sample data set to obtain a support set.

Step 1506: Input the support set and the query set into an initial drug resistance classification model, and input each support sample feature vector and each query sample feature vector into an initial feature screening network through the initial drug resistance classification model.

Step 1508: Perform drug resistance-related feature screening based on each support sample feature vector and each query sample feature vector through the initial feature screening network, to obtain each target support feature vector and each target query feature vector, and input each target support feature vector into the sample screening network.

Step 1510: Map each target support feature vector through the sample screening network to obtain each mapping feature vector, obtain an initial confidence calculation parameter, and perform calculation by using the initial confidence calculation parameter based on each mapping feature vector to obtain a confidence corresponding to each mapping feature vector, and weight each mapping feature vector based on the confidence to obtain each weighted feature vector, and input each weighted feature vector into the prototype network.

Step 1512: Calculate the initial category representation vector corresponding to the drug resistance category based on each weighted feature vector through the prototype network, and determine the training drug resistance category information corresponding to each query sample feature vector based on the similarity degree between each target query feature vector and the initial category representation vector.

Step 1514: Perform logarithmic loss calculation based on the training drug resistance category information and the corresponding drug resistance category label to obtain initial training loss information, and calculate a gradient of the initial training loss information, and reverse the initial drug resistance classification model based on the gradient to obtain an updated drug resistance classification model.

Step 1516: Use the updated drug resistance classification model as the initial drug resistance classification model, return to the step of inputting the support set and the query set into the initial drug resistance classification model, and until a training completion condition is met, use an initial drug resistance classification model when the training completion condition is met as the target drug resistance classification model.

Step 1518: Use the target drug resistance classification model as the initial drug resistance classification model, return to perform the step of extracting from the sample data set to obtain the query set, and until a final training completion condition is met, use an initial drug resistance classification model when the final training completion condition is met as a final drug resistance classification model.

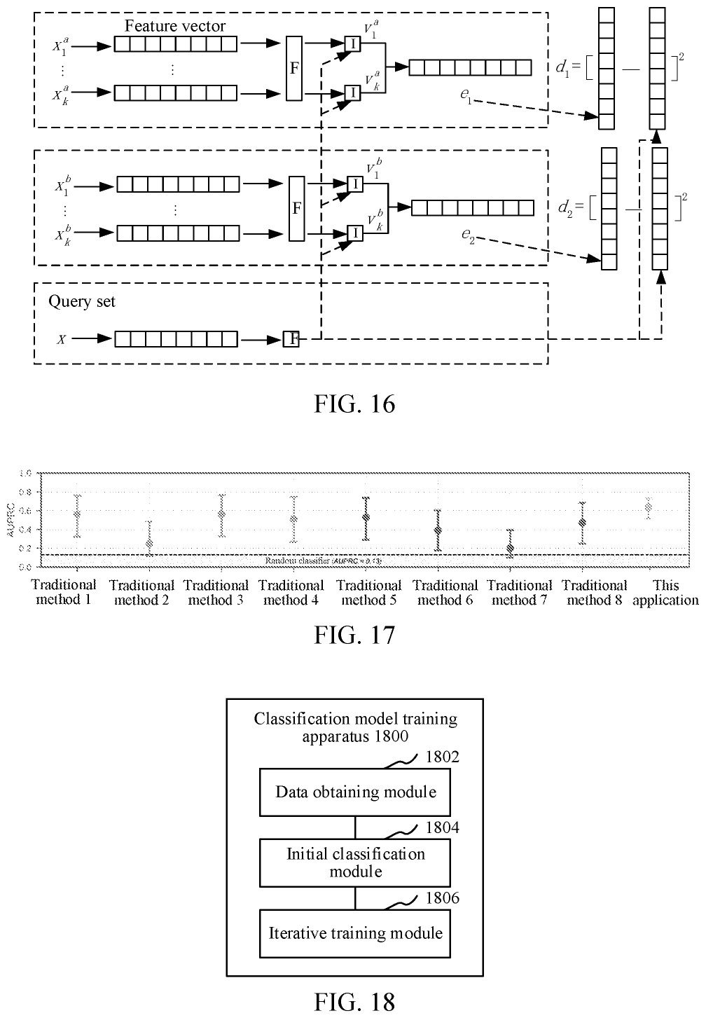

This disclosure further provides an application scenario, which applies the classification model training method. Specifically, as shown in FIG. 16, which is an architectural schematic diagram of drug resistance classification model training, the server extracts a query set and a support set from a sample data set. The support set includes training samples corresponding to the drug-resistant category and training samples corresponding to the non-drug-resistant category, and there are K training samples (x₁^{a}, . . . , xₖ^{a}) and (x₁^{b}, . . . , xₖ^{b}) corresponding to each category. The query set includes training samples x corresponding to each drug resistance category. F refers to a feature screening network, and I refers to a sample screening network. Network parameters are all initialized and need to be trained. That is, the server inputs support feature vectors of the training samples in the support set into the feature screening network of the initial drug resistance classification model, to obtain an outputted target support feature vector, and inputs the target support feature vector into the sample screening network for sample screening, that is, using a confidence calculation parameter V for sample screening, to obtain an outputted weighted feature vector, and calculates an initial category representation vector e corresponding to the drug resistance category based on each weighted feature vector. The initial category representation vectors e include an initial category representation vector e₁ corresponding to the drug-resistant category and an initial category representation vector e₂ corresponding to the non-drug-resistant category. Then, the server calculates a distance d between the initial category representation vector and the target query feature vector obtained through the feature screening network in the query set by the Euclidean distance algorithm, determines the drug resistance category obtained by training according to the distance, then calculates an error between the drug resistance category and the corresponding drug resistance category label through a logarithmic loss function, updates the initial drug resistance classification model reversely according to the error, and obtains the target drug resistance classification model until the training is completed. In this case, an episodic task is completed once, and then a next episodic task is performed. That is, the target drug resistance classification model is used as the initial drug resistance classification model, and the support set and the query set are extracted from the training sample set, to perform loop iteration. Until all episodic tasks are completed, a final drug resistance classification model is obtained.

Then, the drug resistance classification model may be deployed to a server equipped with a Linux operating system or a Windows operating system and central processing unit (CPU) computing resources based on the python (computer programming language) language and the pytorch (an open source Python machine learning library, which is based on Torch and used for applications such as natural language processing) library.

Furthermore, a comparison test may be performed on the final drug resistance classification model obtained by training. Specifically,

the server uses a drug resistance standard data set Platinum and TKI (a class of compounds that can inhibit tyrosine kinase activity) for testing. The server performs feature extraction on drug resistance data in the data set Platinum and TKI to obtain a sample data set. The extracted sample data set is shown in Table 1 below. Non-physical model tools such as RDKit (Open Access Cheminformatics and Machine Learning Toolkit), Biopython (Bioinformatics Resource Library), FoldX (Molecular Simulation Tool), PLIP (Analysis Tool for Protein-Ligand Non-Covalent Interaction), AutoDock (Molecular Simulation Software) are used to generate features with reference value for predicting the change of binding free energy after protein mutation. Rosetta, a hybrid physical and empirical potential energy modeling program, is further used to calculate the energy feature.

TABLE 1
Sample data set

| | | | | | Physical and | |
|---|---|---|---|---|---|---|
| | | Non-drug- | Drug- | | empirical | Total |
| | Quantity | resistant | resistant | Non-physical | potential | quantity |
| Data set | of sample | category | category | model feature | energy feature | of feature |
| Platinum | 4484 | 3362 | 1109 | 1129 | 119 | 1148 |
| TKI | 1144 | 1125 | 119 | 1129 | 119 | 1148 |

There are 148 kinds of sample features in the sample data set, including 129 kinds of non-physical model features and 19 kinds of physical and empirical potential energy features. Then, the support set and the query set are extracted from the sample data set. The support set and the query set extracted in a training process and a test process are shown in Table 2 below.

TABLE 2
Sample extraction table

| Training stage | Support set | Query set |
|---|---|---|
| Model training | 2-way 5-shot | 2-way 5-shot |
| | (Platinum data set) | (Platinum data set) |
| Model validation | 2-way 5-shot | 2-way 5-shot |
| | (Platinum data set) | TKI data set 10 samples |
| | (top 5%, multiple sample | |
| | similarity sampling) | |
| Model testing | 2-way 5-shot | TKI data set |
| | (Platinum data set) | |
| | (top 5%, sample | |
| | similarity sampling) | |

In a drug resistance classification model training (Meta-training) process, the support set and the query set are extracted from the Platinum data set according to a method of 2-way 5-shot, that is, 5 samples of the drug-resistant category are extracted, and 5 samples of the non-drug-resistant category are extracted. In a drug resistance classification model validation (Meta-validation) process, the support set and the query set are extracted from the Platinum data set according to the extraction method of 2-way 5-shot, and 10 samples are extracted from the TKI data set to be also used as the query set to verify the drug resistance classification model. In a drug resistance classification model testing (Meta-testing) process, each sample in the TKI data set is used as sample data to be tested, that is, as the query set in the testing process, and the support set in the testing process is extracted from the Platinum data set according to the extraction method of 2-way 5-shot. Then, the extracted test data is used to test the traditional method and this disclosure. The traditional method may be a method based on molecular dynamics, a traditional machine learning method, or the like. The obtained test evaluation index table is shown as Table 3 below.

TABLE 3
Test evaluation index table

| | | AUPRC | | |
|---|---|---|---|---|
| | Force field | Average | Minimum | Maximum |
| Method | or scoring function | value | value | value |
| Traditional | OPLS3 | 0.56 | 0.32 | 0.76 |
| method 1 | | | | |
| Traditional | Charmm22* and | 0.25 | 0.12 | 0.48 |
| method 2 | CGenFF v 3.0.1 | | | |
| Traditional | Amber99sb*-ILDN | 0.56 | 0.32 | 0.77 |
| method 3 | and GAFF v 2.1 | | | |
| Traditional | Amber99sb*-ILDN | 0.51 | 0.27 | 0.75 |
| method 4 | and GAFF v 2.1 | | | |
| Traditional | REF15 | 0.53 | 0.29 | 0.74 |
| method 5 | | | | |
| Traditional | β NOV16 | 0.39 | 0.18 | 0.60 |
| method 6 | | | | |
| Traditional | n/a | 0.20 | 0.1 | 0.39 |
| method 7 | | | | |
| This disclosure | n/a | 0.61 | 0.51 | 0.71 |

An area under precision recall curve (AUPRC) is used as an evaluation index of the test. It can be clearly seen that an average value and a minimum value of the test evaluation index AUPRC of this disclosure are all superior to that of other traditional methods, and the performance of drug resistance classification and identification is more stable under variance, that is, this disclosure can further improve the accuracy of drug resistance classification and identification. As shown in FIG. 17, which is a specific schematic diagram of the test evaluation index AUPRC, a value of the test evaluation index AUPRC is 0.13 when a method of random classification is used for classification. As can be clearly seen from FIG. 17, the performance of classification and identification in this disclosure is more stable, and the accuracy of drug resistance classification and identification can be further improved.

It is to be understood that, although each step of the flowcharts in FIG. 2 to FIG. 15 is displayed sequentially according to arrows, the steps are not necessarily performed according to an order indicated by arrows. Unless clearly specified in this specification, there is no strict sequence limitation on the execution of the steps, and the steps may be performed in another sequence. Moreover, at least some steps in FIG. 2 to FIG. 15 may include a plurality of steps or a plurality of stages. These steps or the stages are not necessarily performed at the same moment, but may be performed at different moments. The steps or the stages are not necessarily performed in sequence, but may be performed in turn or alternately with another step or at least some of steps or stages of the another step.

In an embodiment, as shown in FIG. 18, a classification model training apparatus 1800 is provided. The apparatus may adopt a software module or a hardware module, or a combination of the two to become a part of the computer device. The apparatus specifically includes: a data obtaining module 1802, an initial classification module 1804, and an iterative training module 1806.

Here, the term module (and other similar terms such as unit, submodule, etc.) may refer to a software module, a hardware module, or a combination thereof. A software module (e.g., computer program) may be developed using a computer programming language. A hardware module may be implemented using processing circuitry and/or memory. Each module can be implemented using one or more processors (or processors and memory). Likewise, a processor (or processors and memory) can be used to implement one or more modules. Moreover, each module can be part of an overall module that includes the functionalities of the module. A module is configured to perform functions and achieve goals such as those described in this disclosure, and may work together with other related modules, programs, and components to achieve those functions and goals.

The data obtaining module 1802 is configured to obtain a support set and a query set, the support set including each support sample feature vector and a corresponding drug resistance category label, and the query set including each query sample feature vector and a corresponding drug resistance category label.

The initial classification module 1804 is configured to input the support set and the query set into an initial drug resistance classification model, perform drug resistance-related feature screening on each support sample feature vector and each query sample feature vector through the initial drug resistance classification model, to obtain each target support feature vector and each target query feature vector, calculate an initial category representation vector corresponding to a drug resistance category based on each target support feature vector, and determine training drug resistance category information corresponding to each query sample feature vector based on a similarity degree between each target query feature vector and the initial category representation vector.

The iterative training module 1806 is configured to update the initial drug resistance classification model based on the training drug resistance category information and the corresponding drug resistance category label, return to perform the step of inputting the support set and the query set into the initial drug resistance classification model, and obtain a target drug resistance classification model when the training is completed, the target drug resistance classification model being used for identifying a drug resistance category corresponding to protein-compound binding.

In an embodiment, the data obtaining module 1802 includes:

a sample obtaining module, configured to obtain a sample data set, the sample data set including a sample feature vector and a drug resistance category label corresponding to each training sample, the sample feature vector being obtained by feature extraction performed based on the training sample, and the training sample including wild-type protein information, mutant protein information, and compound information; and

an extraction module, configured to extract the support set and the query set from the sample data set in different manners.

In an embodiment, the classification model training apparatus 1800 further includes:

a final model training module, configured to use the target drug resistance classification model as the initial drug resistance classification model, return to perform the step of extracting the support set and the query set from the sample data set, and until a final training completion condition is met, use an initial drug resistance classification model when the final training completion condition is met as a final drug resistance classification model.

In an embodiment, the classification model training apparatus 1800 further includes:

a feature extraction module, configured to obtain a training sample, the training sample including wild-type protein information, mutant protein information, and compound information, perform wild feature extraction based on the wild-type protein information and the compound information to obtain a wild feature vector, perform mutation feature extraction based on the mutant protein information and the compound information to obtain a mutation feature vector, and obtain a sample feature vector corresponding to the training sample based on the wild feature vector and the mutation feature vector.

In an embodiment, the extraction module is further configured to perform sampling on the sample data set to obtain the query set in different manners, calculate a similarity degree between each query sample feature vector in the query set and each sample feature vector in the sample data set, sort each sample feature vector in the sample data set based on the similarity degree to obtain a sample feature vector sequence, sequentially select a preset quantity of sample feature vectors from the sample feature vector sequence to obtain an extraction sample data set, and perform extraction on the extraction sample data set in different manners to obtain a support set.

In an embodiment, the initial classification module 1804 is further configured to obtain an initial feature screening parameter, perform the drug resistance-related feature screening on each support sample feature vector respectively based on the initial feature screening parameter to obtain each target support feature vector, and perform the drug resistance-related feature screening on each query sample feature vector based on the initial feature screening parameter to obtain each target query feature vector.

In an embodiment, the initial classification module 1804 is further configured to map each target support feature vector to obtain each mapping feature vector, obtain an initial confidence calculation parameter, and perform calculation by using the initial confidence calculation parameter based on each mapping feature vector to obtain a confidence corresponding to each mapping feature vector, weight each mapping feature vector based on the confidence to obtain each weighted feature vector, and calculate the initial category representation vector corresponding to the drug resistance category based on each weighted feature vector.

In an embodiment, the drug resistance category includes a drug-resistant category and a non-drug-resistant category.

The initial classification module 1804 is further configured to divide each weighted feature vector according to the drug resistance category label corresponding to each support sample feature vector to obtain a weighted feature vector corresponding to the drug-resistant category and a weighted feature vector corresponding to the non-drug-resistant category, perform vector averaging based on the weighted feature vector corresponding to the drug-resistant category to obtain a first initial category representation vector corresponding to the drug-resistant category, and perform vector averaging based on the weighted feature vector corresponding to the non-drug-resistant category to obtain a second initial category representation vector corresponding to the non-drug-resistant category.

In an embodiment, the initial classification module 1804 is further configured to calculate a distance between a current target query feature vector and the first initial category representation vector and a distance between the current target query feature vector and the second initial category representation vector, to obtain a current first initial distance and a current second initial distance, and compare the current first initial distance with the current second initial distance, training drug resistance category information corresponding to the current target query feature vector being the non-drug-resistant category when the current first target distance exceeds the current second target distance, and the training drug resistance category information corresponding to the current target query feature vector being the drug-resistant category when the current first target distance does not exceed the current second target distance.

In an embodiment, the initial drug resistance classification model includes an initial feature screening network and an initial classification network. The initial classification module 1804 is further configured to input the support set and the query set into the initial drug resistance classification model, and input each support sample feature vector and each query sample feature vector into the initial feature screening network through the initial drug resistance classification model, perform the drug resistance-related feature screening based on each support sample feature vector and each query sample feature vector through the initial feature screening network, to obtain each target support feature vector and each target query feature vector, and input each target support feature vector and each target query feature vector into the classification network, and calculate the initial category representation vector corresponding to the drug resistance category based on each target support feature vector through the classification network, and determine the training drug resistance category information corresponding to each query sample feature vector based on the similarity degree between each target query feature vector and the initial category representation vector.

In an embodiment, the classification network includes a sample screening network and a prototype network. The initial classification module 1804 is further configured to input each target support feature vector into the sample screening network, and map each target support feature vector through the sample screening network to obtain each mapping feature vector, obtain an initial confidence calculation parameter, and perform calculation by using the initial confidence calculation parameter based on each mapping feature vector to obtain a confidence corresponding to each mapping feature vector, weight each mapping feature vector based on the confidence to obtain each weighted feature vector, and input each weighted feature vector into the prototype network, and calculate the initial category representation vector corresponding to the drug resistance category based on each weighted feature vector through the prototype network, and determine the training drug resistance category information corresponding to each query sample feature vector based on the similarity degree between each target query feature vector and the initial category representation vector.

In an embodiment, the iterative training module 1806 is further configured to perform logarithmic loss calculation based on the training drug resistance category information and the corresponding drug resistance category label to obtain initial training loss information, calculate a gradient of the initial training loss information, and reverse the initial drug resistance classification model based on the gradient to obtain an updated drug resistance classification model, and use the updated drug resistance classification model as the initial drug resistance classification model, return to the step of inputting the support set and the query set into the initial drug resistance classification model, and until a training completion condition is met, use an initial drug resistance classification model when the training completion condition is met as the target drug resistance classification model.

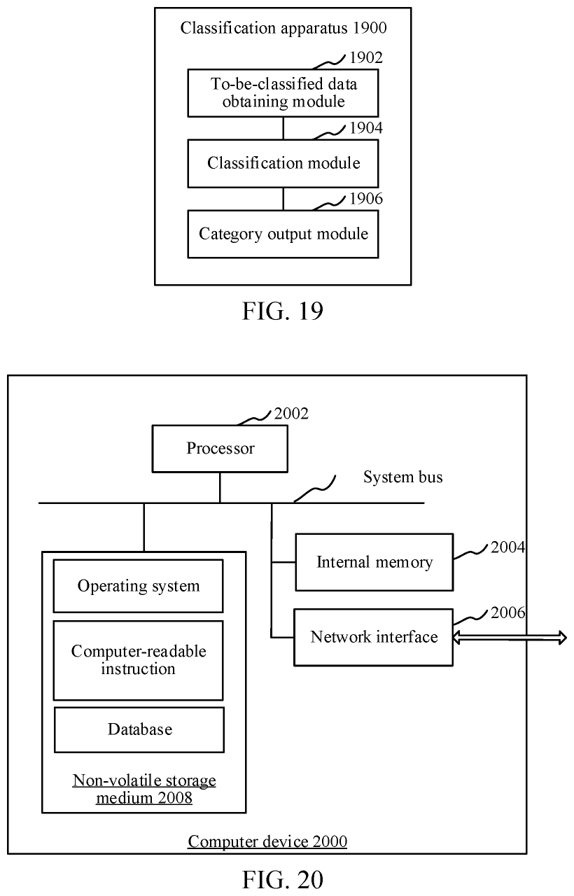

In an embodiment, as shown in FIG. 19, a classification apparatus 1900 is provided. The apparatus may adopt a software module or a hardware module, or a combination of the two to become a part of the computer device. The apparatus specifically includes: a original classification data obtaining module 1902, a classification module 1904, and a category output module 1906.

The original classification data obtaining module 1902 is configured to obtain original classification data and sample data, the original classification data including original classification feature vectors, and the sample data including each sample feature vector and a corresponding sample category label.

The classification module 1904 is configured to input the original classification data and the sample data into a drug resistance classification model, perform drug resistance-related feature screening based on the original classification feature vector and each sample feature vector through the drug resistance classification model, to obtain a target original classification feature vector and each target sample feature vector, calculate a target category representation vector corresponding to a sample category based on each target sample feature vector, and determine drug resistance category information corresponding to the original classification feature vector based on a similarity degree between the target original classification feature vector and the target category representation vector.

The category output module 1906 is configured to output the drug resistance category information corresponding to the original classification data through the drug resistance classification model.

For a specific limitation on the classification model training apparatus and the classification apparatus, refer to the limitation on the classification model training method and the classification method above. The modules in the classification model training apparatus and the classification apparatus may be implemented entirely or partially by software, hardware, or a combination thereof. The modules may be built in or independent of a processor of a computer device in a hardware form, or may be stored in a memory of the computer device in a software form, so that the processor invokes and performs an operation corresponding to each of the modules.

In an embodiment, a computer device is provided. The computer device may be a server, and an internal structure diagram thereof may be shown in FIG. 20. The computer device 2000 includes a processor 2002, a memory, and a network interface 2006 that are connected through a system bus. The processor 2002 of the computer device 2000 is configured to provide computing and control capabilities. The memory of the computer device 2000 includes a non-volatile storage medium 2008 and an internal memory 2004. The non-volatile storage medium 2008 stores an operating system, computer-readable instructions, and a database. The internal memory 2004 provides an environment for running of the operating system and the computer-readable instructions in the non-volatile storage medium. The database of the computer device is configured to store training sample data. The network interface 2006 of the computer device 2000 is configured to communicate with an external terminal through a network connection. The computer-readable instructions, when executed by the processor, implement a classification model training method and a classification method.

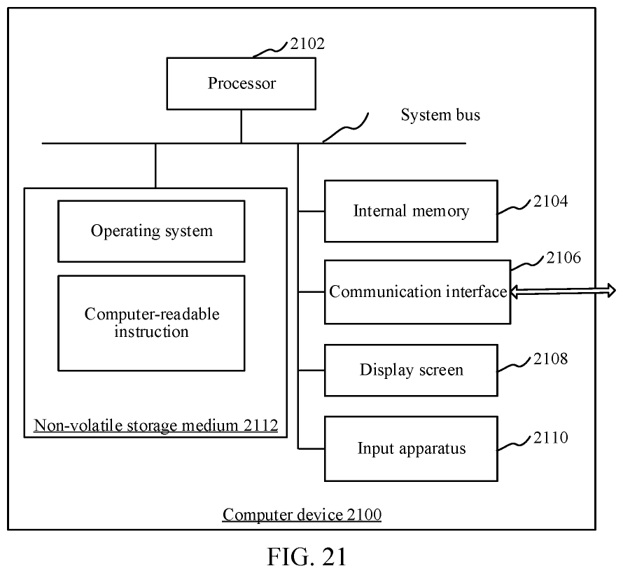

In an embodiment, a computer device is provided. The computer device may be a terminal, and an internal structure diagram thereof may be shown in FIG. 21. The computer device 2100 includes a processor 2102, a memory, a communication interface 2106, a display screen 2108, and an input apparatus 2110 that are connected through a system bus. The processor 2102 of the computer device 2100 is configured to provide computing and control capabilities. The memory of the computer device includes a non-volatile storage medium 2112 and an internal memory 2102. The non-volatile storage medium 2112 stores an operating system and computer-readable instructions. The internal memory 2102 provides an environment for running of the operating system and the computer-readable instructions in the non-volatile storage medium. The communication interface 2106 of the computer device 2100 is configured to communicate with an external terminal in a wired or wireless manner, and the wireless manner may be implemented by WIFI, an operator network, near field communication (NFC) or other technologies. The computer-readable instructions, when executed by the processor, implement a classification model training method and a classification method. The display screen 2108 of the computer device 2100 may be a liquid crystal display screen or an electronic ink display screen. The input apparatus 2110 of the computer device 2100 may be a touch layer covering the display screen, or may be a key, a trackball, or a touch pad disposed on a housing of the computer device, or may be an external keyboard, a touch pad, a mouse, or the like.

A person skilled in the art may understand that the structure shown in FIG. 19 and FIG. 20 is merely a block diagram of a part of the structure related to the solution of this disclosure, and does not constitute a limitation on a computer device to which the solution of this disclosure is applied. In particular, the computer device may include more or fewer components than those shown in the figure, or some components may be combined, or a different component deployment may be used.

In an embodiment, a computer device is provided, including a memory and a processor, the memory storing computer-readable instructions, the processor, when executing the computer-readable instructions, implementing the steps in the foregoing method embodiments.

In an embodiment, a computer-readable storage medium is provided, storing computer-readable instructions, the computer-readable instructions, when executed by a processor, implementing the steps in the foregoing method embodiments.

In an embodiment, a computer program product or a computer program is provided, the computer program product or the computer program including computer instructions, the computer instructions being stored in a computer-readable storage medium. A processor of the computer device reads the computer instructions from the computer-readable storage medium, and the processor executes the computer instructions, to cause the computer device to perform the steps in the foregoing method embodiments.

A person of ordinary skill in the art may understand that all or some of the procedures of the methods of the foregoing embodiments may be implemented by computer-readable instructions instructing relevant hardware. The computer-readable instructions may be stored in a non-volatile computer-readable storage medium. When the computer-readable instructions are executed, the procedures of the embodiments of the foregoing methods may be included. Any reference to a memory, a storage, a database, or another medium used in the embodiments provided in this disclosure may include at least one of a non-volatile memory and a volatile memory. The non-volatile memory may include a read-only memory (ROM), a magnetic tape, a floppy disk, a flash memory, an optical memory, and the like. The volatile memory may include a random access memory (RAM) or an external cache. For the purpose of description instead of limitation, the RAM is available in a plurality of forms, such as a static random access memory (SRAM) or a dynamic random access memory (DRAM).

The technical features in the foregoing embodiments may be randomly combined. For concise description, not all possible combinations of the technical features in the embodiments are described. However, provided that combinations of the technical features do not conflict with each other, the combinations of the technical features are considered as falling within the scope described in this specification.

The foregoing embodiments only describe several implementations of this disclosure, which are described specifically and in detail, but cannot be construed as a limitation to the patent scope of this disclosure. For a person of ordinary skill in the art, several transformations and improvements can be made without departing from the idea of this disclosure. These transformations and improvements belong to the protection scope of this disclosure. Therefore, the protection scope of the patent of this disclosure shall be subject to the appended claims.

What is claimed is:

1. A classification model training method, performed by a computer device, the method comprising:
obtaining a support set and a query set, the support set comprising support sample feature vectors and corresponding drug resistance category labels, and the query set comprising query sample feature vectors and corresponding drug resistance category labels;
inputting the support set and the query set into an initial drug resistance classification model;
performing drug resistance-related feature screening on the support sample feature vectors and the query sample feature vectors through the initial drug resistance classification model, to obtain target support feature vectors and target query feature vectors;
calculating an initial category representation vector corresponding to a drug resistance category based on the target support feature vectors;
determining training drug resistance category information corresponding to the query sample feature vectors based on a similarity degree between the target query feature vectors and the initial category representation vector;
updating the initial drug resistance classification model based on the training drug resistance category information and the corresponding drug resistance category labels;
returning to perform the operation of inputting the support set and the query set into the initial drug resistance classification model; and
obtaining a target drug resistance classification model in response to training being completed, the target drug resistance classification model being for identifying a drug resistance category corresponding to protein-compound binding.

2. The method according to claim 1, wherein the obtaining the support set and the query set comprises:
obtaining a sample data set, the sample data set comprising sample feature vectors and drug resistance category labels corresponding to training samples, the sample feature vectors being obtained by feature extraction performed based on the training samples; and
extracting the support set and the query set from the sample data set.

3. The method according to claim 2, wherein the method further comprises:
using the target drug resistance classification model as the initial drug resistance classification model;
returning to perform the operation of extracting the support set and the query set from the sample data set; and
in response to a final training completion condition being met, using the initial drug resistance classification model as a final drug resistance classification model.

4. The method according to claim 2, wherein the method further comprises:
obtaining a training sample, the training sample comprising wild-type protein information, mutant protein information, and compound information;
performing wild feature extraction based on the wild-type protein information and the compound information to obtain a wild feature vector;
performing mutation feature extraction based on the mutant protein information and the compound information to obtain a mutation feature vector; and
obtaining a sample feature vector corresponding to the training sample based on the wild feature vector and the mutation feature vector.

5. The method according to claim 2, wherein the extracting the support set and the query set from the sample data set comprises:
performing sampling on the sample data set to obtain the query set;
calculating a similarity degree between the query sample feature vectors in the query set and the sample feature vectors in the sample data set;
sorting the sample feature vectors in the sample data set based on the similarity degree to obtain a sample feature vector sequence;
sequentially selecting a preset quantity of sample feature vectors from the sample feature vector sequence to obtain an extraction sample data set; and
performing extraction on the extraction sample data set to obtain the support set.

6. The method according to claim 1, wherein the performing the drug resistance-related feature screening on the support sample feature vectors and the query sample feature vectors to obtain the target support feature vectors and the target query feature vectors comprises:
obtaining an initial feature screening parameter;
performing the drug resistance-related feature screening on the support sample feature vectors respectively based on the initial feature screening parameter to obtain the target support feature vectors; and
performing the drug resistance-related feature screening on the query sample feature vectors respectively based on the initial feature screening parameter to obtain the target query feature vectors.

7. The method according to claim 1, wherein the calculating the initial category representation vector corresponding to the drug resistance category based on the target support feature vectors comprises:
mapping the target support feature vectors to obtain mapping feature vectors;
obtaining an initial confidence calculation parameter, and performing calculation with the initial confidence calculation parameter based on the mapping feature vector to obtain a confidence corresponding to the mapping feature vectors;
weighting the mapping feature vectors based on the confidence to obtain weighted feature vectors; and
calculating the initial category representation vector corresponding to the drug resistance category based on the weighted feature vectors.

8. The method according to claim 7, wherein the drug resistance category comprises a drug-resistant category and a non-drug-resistant category, and the calculating the initial category representation vector corresponding to the drug resistance category based on the weighted feature vectors comprises:
dividing the weighted feature vectors according to the drug resistance category labels corresponding to the support sample feature vectors to obtain a weighted feature vector corresponding to the drug-resistant category and a weighted feature vector corresponding to the non-drug-resistant category;
performing vector averaging based on the weighted feature vector corresponding to the drug-resistant category to obtain a first initial category representation vector corresponding to the drug-resistant category; and
performing vector averaging based on the weighted feature vector corresponding to the non-drug-resistant category to obtain a second initial category representation vector corresponding to the non-drug-resistant category.

9. The method according to claim 8, wherein the determining the training drug resistance category information corresponding to the query sample feature vectors based on the similarity degree between the target query feature vectors and the initial category representation vector comprises:
calculating a distance between a current target query feature vector in the target query feature vectors and the first initial category representation vector and a distance between the current target query feature vector in the target query feature vectors and the second initial category representation vector, to obtain a current first initial distance and a current second initial distance; and
comparing the current first initial distance with the current second initial distance, training drug resistance category information corresponding to the current target query feature vector being the non-drug-resistant category in response to the current first initial distance exceeding the current second initial distance, and the training drug resistance category information corresponding to the current target query feature vector being the drug-resistant category in response to the current first initial distance failing to exceed the current second initial distance.

10. The method according to claim 1, wherein the initial drug resistance classification model comprises an initial feature screening network and an initial classification network, and the inputting the support set and the query set into the initial drug resistance classification model comprises:
inputting the support set and the query set into the initial drug resistance classification model, and inputting the support sample feature vectors and the query sample feature vectors into the initial feature screening network through the initial drug resistance classification model;
performing the drug resistance-related feature screening based on the support sample feature vectors and the query sample feature vectors through the initial feature screening network, to obtain the target support feature vectors and the target query feature vectors;
inputting the target support feature vectors and the target query feature vectors into the classification network;
calculating the initial category representation vector corresponding to the drug resistance category based on the target support feature vectors through the classification network; and
determining the training drug resistance category information corresponding to the query sample feature vectors based on the similarity degree between the target query feature vectors and the initial category representation vector.

11. The method according to claim 10, wherein the classification network comprises a sample screening network and a prototype network, and the inputting the target support feature vector and the target query feature vector into the classification network comprises:
inputting the target support feature vector into the sample screening network;
mapping the target support feature vectors through the sample screening network to obtain mapping feature vectors;
obtaining an initial confidence calculation parameter;
performing calculation using the initial confidence calculation parameter based on the mapping feature vectors to obtain a confidence corresponding to the mapping feature vectors;
weighting the mapping feature vectors based on the confidence to obtain weighted feature vectors;
inputting the weighted feature vectors into the prototype network;
calculating the initial category representation vector corresponding to the drug resistance category based on the weighted feature vectors through the prototype network; and
determining the training drug resistance category information corresponding to the query sample feature vectors based on the similarity degree between the target query feature vectors and the initial category representation vector.

12. The method according to claim 1, wherein the updating the initial drug resistance classification model based on the training drug resistance category information and the corresponding drug resistance category labels comprises:
performing logarithmic loss calculation based on the training drug resistance category information and the corresponding drug resistance category label to obtain initial training loss information;
calculating a gradient of the initial training loss information; and
reversing the initial drug resistance classification model based on the gradient to obtain an updated drug resistance classification model;
the returning to perform the operation of inputting the support set and the query set into the initial drug resistance classification model comprises:
using the updated drug resistance classification model as the initial drug resistance classification model;
returning to the operation of inputting the support set and the query set into the initial drug resistance classification model;
the obtaining the target drug resistance classification model in response to the training being completed comprises:
in response to a training completion condition being met, using an initial drug resistance classification model as the target drug resistance classification model.

13. A classification method, performed by a computer device, the method comprising:
obtaining original classification data and sample data, the original classification data comprising original classification feature vectors, and the sample data comprising sample feature vectors and corresponding sample category labels;
inputting the original classification data and the sample data into a drug resistance classification model;
performing drug resistance-related feature screening based on the original classification feature vectors and the sample feature vectors through the drug resistance classification model, to obtain a target original classification feature vector and target sample feature vectors;
calculating a target category representation vector corresponding to a sample category based on the target sample feature vectors;
determining drug resistance category information corresponding to the original classification feature vector based on a similarity degree between the target original classification feature vector and the target category representation vector; and
outputting the drug resistance category information corresponding to the original classification data through the drug resistance classification model.

14. A classification model training apparatus, comprising:
a memory operable to store computer-readable instructions; and
a processor circuitry operable to read the computer-readable instructions, the processor circuitry when executing the computer-readable instructions is configured to:
obtain a support set and a query set, the support set comprising support sample feature vectors and corresponding drug resistance category labels, and the query set comprising query sample feature vectors and corresponding drug resistance category labels;
input the support set and the query set into an initial drug resistance classification model;
perform drug resistance-related feature screening on the support sample feature vectors and the query sample feature vectors through the initial drug resistance classification model, to obtain target support feature vectors and target query feature vectors;
calculate an initial category representation vector corresponding to a drug resistance category based on the target support feature vectors;
determine training drug resistance category information corresponding to the query sample feature vectors based on a similarity degree between the target query feature vectors and the initial category representation vector;
update the initial drug resistance classification model based on the training drug resistance category information and the corresponding drug resistance category labels;
return to perform the operation of inputting the support set and the query set into the initial drug resistance classification model; and
obtain a target drug resistance classification model in response to training being completed, the target drug resistance classification model being for identifying a drug resistance category corresponding to protein-compound binding.

15. The apparatus according to claim 14, wherein the processor circuitry is configured to:
obtain a sample data set, the sample data set comprising sample feature vectors and drug resistance category labels corresponding to training samples, the sample feature vectors being obtained by feature extraction performed based on the training samples; and
extract the support set and the query set from the sample data set.

16. The apparatus according to claim 14, wherein the processor circuitry is configured to:
obtain an initial feature screening parameter;
perform the drug resistance-related feature screening on the support sample feature vectors respectively based on the initial feature screening parameter to obtain the target support feature vectors; and
perform the drug resistance-related feature screening on the query sample feature vectors respectively based on the initial feature screening parameter to obtain the target query feature vectors.

17. The apparatus according to claim 14, wherein the processor circuitry is configured to:
map the target support feature vectors to obtain mapping feature vectors;
obtain an initial confidence calculation parameter, and perform calculation with the initial confidence calculation parameter based on the mapping feature vector to obtain a confidence corresponding to the mapping feature vectors;
weight the mapping feature vectors based on the confidence to obtain weighted feature vectors; and
calculate the initial category representation vector corresponding to the drug resistance category based on the weighted feature vectors.

18. The apparatus according to claim 14, wherein the initial drug resistance classification model comprises an initial feature screening network and an initial classification network, and the processor circuitry is configured to:
input the support set and the query set into the initial drug resistance classification model, and input the support sample feature vectors and the query sample feature vectors into the initial feature screening network through the initial drug resistance classification model;
perform the drug resistance-related feature screening based on the support sample feature vectors and the query sample feature vectors through the initial feature screening network, to obtain the target support feature vectors and the target query feature vectors;
input the target support feature vectors and the target query feature vectors into the classification network;
calculate the initial category representation vector corresponding to the drug resistance category based on the target support feature vectors through the classification network; and
determine the training drug resistance category information corresponding to the query sample feature vectors based on the similarity degree between the target query feature vectors and the initial category representation vector.

19. The apparatus according to claim 18, wherein the classification network comprises a sample screening network and a prototype network, and the processor circuitry is configured to:
input the target support feature vector into the sample screening network;
map the target support feature vectors through the sample screening network to obtain mapping feature vectors;
obtain an initial confidence calculation parameter;
perform calculation using the initial confidence calculation parameter based on the mapping feature vectors to obtain a confidence corresponding to the mapping feature vectors;
weight the mapping feature vectors based on the confidence to obtain weighted feature vectors;
input the weighted feature vectors into the prototype network;
calculate the initial category representation vector corresponding to the drug resistance category based on the weighted feature vectors through the prototype network; and
determine the training drug resistance category information corresponding to the query sample feature vectors based on the similarity degree between the target query feature vectors and the initial category representation vector.

20. The apparatus according to claim 14, wherein the processor circuitry is configured to:
perform logarithmic loss calculation based on the training drug resistance category information and the corresponding drug resistance category label to obtain initial training loss information;
calculate a gradient of the initial training loss information;
reverse the initial drug resistance classification model based on the gradient to obtain an updated drug resistance classification model;
use the updated drug resistance classification model as the initial drug resistance classification model;
return to the operation of inputting the support set and the query set into the initial drug resistance classification model; and
in response to a training completion condition being met, use an initial drug resistance classification model as the target drug resistance classification model.
